(19) United States
(12) Patent Application Publication
(10) Pub. No.: US 2019/0069771 A1
(43) Pub. Date: Mar. 7, 2019

(54) VIBRATING DENTAL MIRROR

(71) Applicant: Robert SHIMONOV, Petah Tikva (IL)

(72) Inventor: Robert SHIMONOV, Petah Tikva (IL)

(21) Appl. No.: 16/181,483

(22) Filed: Nov. 6, 2018

Related U.S. Application Data
(63) Continuation-in-part of application No. PCT/IB2017/058465, filed on Dec. 28, 2017.
(60) Provisional application No. 62/441,384, filed on Jan. 1, 2017.

(51) Int. Cl. A61B 1/247; A61C 19/08; A61H 13/00; A61H 23/02

(52) U.S. Cl. CPC A61B 1/247; A61H 23/02; A61H 13/00; A61C 19/08

(57) ABSTRACT

A vibrating dental device, including:
an elongated handle, wherein the handle further includes a vibrator configured to generate vibrations with at least one desired vibration parameter;
an attachment piece connected to the vibrator, wherein an end of the attachment piece is sized and shaped to be placed in contact with a deformable soft tissue of a mouth at a contact site;
a driving circuitry within the elongated handle electrically connected to the vibrator, wherein the driving circuitry delivers electricity to the vibrator to vibrate the attachment piece with at least one vibration parameter selected to reduce sensitivity to pain at a different target site from the contact site, while the attachment piece is in contact with the deformable soft tissue at the contact site.

### RELATED APPLICATIONS

This application is a Continuation-in-Part (CIP) of PCT Patent Application No. PCT/IB2017/058465 filed on Dec. 28, 2017, which claims the benefit of priority under 35 USC § 119(e) of U.S. Provisional Patent Application No. 62/441,384 filed on Jan. 1, 2017. The contents of the above applications are all incorporated by reference as if fully set forth herein in their entirety.

### FIELD AND BACKGROUND OF THE INVENTION

The present invention, in some embodiments thereof, relates to a vibrating device and, more particularly, but not exclusively, to a vibrating device for reducing dental pain.

### SUMMARY OF THE INVENTION

Following are some examples of some embodiments of the invention:

Example 1. A vibrating dental device, comprising:

an elongated handle, wherein said handle further comprises a vibrator configured to generate vibrations with a desired vibration amplitude;

an attachment piece connected to said vibrator, wherein said attachment piece is sized and shaped to be placed in contact with the inner side of a cheek or with the inner side of a lip at a contact site;

wherein a driving circuitry delivers electricity to said vibrator to vibrate said attachment piece with a sufficient vibration amplitude to reduce sensitivity to pain at a different target site, while said attachment piece is in contact with said inner side of said cheek or with said inner side of said lip at said contact site.

Example 2. The device of example 1, wherein said attachment piece is a dental mirror.

Example 3. The device of example 2, wherein said dental mirror contacts said contact site at an angle to allow visualization of said target site.

Example 4. The device of any one of the previous examples, further comprising a control circuitry, wherein said control circuitry controls said vibrator to generate said sufficient vibration amplitude.

Example 5. The device of example 4, wherein said control circuitry controls the delivery of said electricity to said vibrator by said driving circuitry.

Example 6. The device of any of the previous examples, further comprising a memory storage, wherein said memory storage stores at least one vibration protocol.

Example 7. The device of example 6, further comprising an interface for selecting said at least one vibration protocol.

Example 8. The device of example 7, wherein said interface delivers an indication when said at least one vibration protocol reaches a desired stage.

Example 9. The device of any one of examples 1 to 5, further comprising a memory storage, wherein said memory storage stores at least one vibration parameter.

Example 10. The device of example 9, wherein said at least one vibration parameter comprises maximal vibration amplitude, vibration frequency, overall vibration duration and/or vibration duration at maximal vibration amplitude.

Example 11. The device of examples 9 or 10, further comprising an interface for selecting said at least one vibration parameter.

Example 12. The device of example 11, wherein said interface delivers an indication when said at last one vibration parameter reaches a desired value.

Example 13. The device of any of the previous examples, wherein said sufficient vibration amplitude is at least 2 mm.

Example 14. The device of any of the previous examples, wherein said handle further comprises a connector shaped to allow insertion and removal of said attachment piece from said handle.

Example 15. The device of any of the previous examples, wherein said handle further comprises a damper, wherein said damper reduces the vibrations delivered from said vibrator to said handle by at least 50% compared to the vibrations of said attachment piece.

Example 16. The device of any of the previous examples, further comprising a sensor coupled between said attachment piece and said vibrator, to sense a vibration parameter of said attachment piece.

Example 17. The device of example 16, wherein said vibration parameter of said attachment piece comprise vibration amplitude and/or vibration frequency.

Example 18. The device of any of the previous examples, wherein the distance between said contact site to said target site is at least 1 centimeter.

Example 19. The device of any of the previous examples wherein said vibrator vibrates said attachment piece with a vibration frequency in the range of 2-200 Hz.

Example 20. A method for reducing sensitivity to pain, comprising:

placing a dental mirror connected to a vibrating device in contact with an inner side of a cheek or with an inner side of a lip;

vibrating said inner side of said cheek using said dental mirror for reducing sensitivity to pain at a different target site from said cheek.

Example 21. The method of example 20, wherein said vibrating further comprises vibrating said inner side of a cheek or said inner side of a lip according to a vibration protocol.

Example 22. The method of example 21, further comprising selecting said vibration protocol prior to said vibrating.

Example 23. The method of examples 21 or 22, further comprising determining if said vibrating is according to said vibration protocol.

Example 24. The method of example 23, further comprising delivering an indication if said vibrating protocol reaches a desired protocol stage.

Example 25. The method of example 20, wherein said vibrating further comprises vibrating said inner side of a cheek or inner side of a lip according to at least one vibration parameter.

Example 26. The method of example 25, further comprising selecting said at least one vibration parameter.

Example 27. The method of examples 25 or 26, further comprising determining if said vibrating is according to said at least one vibration parameter.

Example 28. The method of example 27, further comprising delivering an indication if said at least one vibration parameter reaches a desired value.

Example 29. The method of any one of examples 20 to 28, further comprising connecting said dental mirror to a handle of said vibrating device prior to said placing.

Example 30. The method of any one of examples 20 to 29, further comprising stretching said inner side of said cheek before and/or during said vibrating.

Following are some additional examples of some embodiments of the invention:

Example 1. A vibrating dental device, comprising:

an elongated handle, wherein said handle further comprises a vibrator configured to generate vibrations with at least one desired vibration parameter;

an attachment piece connected to said vibrator, wherein an end of said attachment piece is sized and shaped to be placed in contact with a deformable soft tissue of a mouth at a contact site;

a driving circuitry within said elongated handle electrically connected to said vibrator, wherein said driving circuitry delivers electricity to said vibrator to vibrate said attachment piece with at least one vibration parameter selected to reduce sensitivity to pain at a different target site from said contact site, while said attachment piece is in contact with said deformable soft tissue at said contact site.

Example 2. The device of example 1, wherein said deformable soft tissue comprises the inner side of a cheek and/or the inner side of a lip.

Example 3. The device of examples 1 or 2, wherein said attachment piece is a dental mirror.

Example 4. The device of example 3, wherein said dental mirror contacts said contact site at an angle to allow visualization of said target site.

Example 5. The device of examples 1 or 2, wherein said selected at least one vibration parameter comprises vibration amplitude and/or vibration frequency.

Example 6. The device of example 5, wherein said vibration amplitude is at least 2 mm.

Example 7. The device of example 5, wherein said vibration frequency is at least 10 Hz.

Example 8. The device of example 1, further comprising a control circuitry, wherein said control circuitry controls said vibrator to generate said vibration according to said at least one selected vibration parameter.

Example 9. The device of example 8, wherein said control circuitry controls the delivery of said electricity to said vibrator by said driving circuitry.

Example 10. The device of example 1, further comprising a memory storage, wherein said memory storage stores at least one vibration protocol and/or at values of least one vibration parameter.

Example 11. The device of example 10, further comprising an interface for selecting said at least one vibration protocol and/or said values of said at least one vibration parameter.

Example 12. The device of example 11, wherein said interface delivers an indication when said at least one vibration protocol reaches a desired stage.

Example 13. The device of example 10, wherein said memory storage stores said at least one selected vibration parameter.

Example 14. The device of example 1, wherein said at least one selected vibration parameter comprises maximal vibration amplitude, overall vibration duration and/or vibration duration at maximal vibration amplitude.

Example 15. The device of example 1, wherein said handle further comprises a connector shaped to allow insertion and removal of said attachment piece from said handle.

Example 16. The device of example 1, wherein said handle further comprises a damper, wherein said damper reduces the vibrations delivered from said vibrator to said handle by at least 50% compared to the vibrations of said attachment piece.

Example 17. The device of example 1, further comprising a sensor coupled between said attachment piece and said vibrator, to sense a vibration parameter of said attachment piece.

Example 18. The device of example 17, wherein said vibration parameter of said attachment piece comprise vibration amplitude and/or vibration frequency.

Example 19. The device of example 1, wherein the distance between said contact site to said target site is at least 1 centimeter.

Example 20. The device of example 1, comprising a rechargeable battery and a charging socket electrically connected to said rechargeable battery.

Example 21. The device of example 1, comprising a first interlocking geometry, and wherein said attachment piece comprises a second interlocking geometry, and wherein said first interlocking geometry and said second interlocking geometry are shaped and sized for mechanically interlocking with each other.

Example 22. The device of example 21, wherein said first interlocking geometry comprises a protrusion and said second interlocking geometry comprises a recess, a window or an opening.

Example 23. The device of example 21, wherein said first interlocking geometry comprises a press-fit plunger and said second interlocking geometry comprises a recess, a window or an opening.

Example 24. The device of example 1, comprising at least one light source, wherein said at least one light source illuminates said contact site and/or said target site.

Example 25. A method for reducing sensitivity to pain, comprising: placing at least part of an attachment piece connected to a vibrating device in contact with a deformable tissue of a mouth at a contact site; vibrating said deformable tissue using said attachment piece for reducing sensitivity to pain at a different target site from said contact site.

Example 26. The method of example 25, wherein said attachment piece comprises a dental mirror.

Example 27. The method of any one of examples 25 or 26, wherein said deformable tissue of the mouth comprises an inner side of a cheek or an inner side of a lip.

Example 28. The method of example 27, wherein said vibrating further comprises vibrating said inner side of a cheek or said inner side of a lip according to a vibration protocol.

Example 29. The method of example 28, further comprising selecting said vibration protocol prior to said vibrating.

Example 30. The method of example 28, further comprising determining if said vibrating is according to said vibration protocol.

Example 31. The method of example 30, further comprising delivering an indication if said vibrating protocol reaches a desired protocol stage.

Example 32. The method of example 25, wherein said vibrating further comprises vibrating said inner side of a cheek or inner side of a lip according to at least one vibration parameter.

Example 33. The method of example 32, further comprising selecting said at least one vibration parameter.

Example 34. The method of examples 32 or 33, further comprising determining if said vibrating is according to said at least one vibration parameter.

Example 35. The method of example 34, further comprising delivering an indication if said at least one vibration parameter reaches a desired value.

Example 36. The method of example 25, further comprising connecting said attachment piece to a handle of said vibrating device prior to said placing.

Example 37. The method of example 27, further comprising stretching said inner side of said cheek before and/or during said vibrating.

Example 38. A dental mirror, comprising:

a mirror;

a shaft having a distal section and a proximal section, wherein said shaft is connected to said mirror at said distal section, and wherein at least part of said proximal section has a non-rotational symmetry shaped and sized to conform to an inner lumen of a handle.

Example 39. The dental mirror of example 38, wherein said proximal section comprises a first interlocking geometry shape and sized to mechanically interlock with a second interlocking geometry in said handle.

Example 40. The dental mirror of example 39, wherein said first interlocking geometry comprises a recess, an opening, and wherein said second interlocking geometry comprises a press-fit plunger or a protrusion.

Example 41. The dental mirror of examples 39 or 40, wherein said first interlocking geometry comprises a protrusion, and wherein said second interlocking geometry comprises a recess, an opening or a window.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Some of the methods described herein are generally designed only for use by a computer, and may not be feasible or practical for performing purely manually, by a human expert. A human expert who wanted to manually perform similar tasks, such as determining vibration parameters, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1C are illustrations of a vibrating dental mirror for reducing pain, placed inside the mouth of a patient, according to some embodiments of the invention;

FIG. 2 is a flow chart depicting a general process for reducing pain sensitivity, according to some embodiments of the invention;

FIG. 3 is a graph depicting the change in vibrating amplitude over time in 3 different activation protocols, according to some embodiments of the invention;

FIG. 4 is a block diagram of a vibrating dental device, according to some embodiments of the invention;

FIG. 5 is a flow chart depicting a process for vibrating the inner side of a cheek and/or the inner side of a lip prior to a dental treatment, according to some embodiments of the invention;

FIGS. 6A-6C are schematic illustrations of a dental mirror connected to a vibrating device, according to some embodiments of the invention;

FIG. 7 is a schematic illustration of a dental mirror connected to a vibrating device placed inside a mouth of a patient, according to some embodiments of the invention;

FIGS. 8A-8F are schematic illustrations of different connections between a dental mirror and the vibrating device, according to some embodiments of the invention;

FIGS. 9A-9D are schematic illustrations of a dental mirror and a handle, FIG. 9C is a cross-section view along line I-I, FIG. 9D is a cross-section view along line H-H, according to some embodiments of the invention;

FIGS. 10A-10E are schematic illustrations of an assembly between a dental mirror with a handle and a vibrating device, according to some embodiments of the invention;

FIGS. 11A-11F are schematic illustrations of an assembly between a dental mirror without a handle and a vibrating device, according to some embodiments of the invention;

FIG. 12 is a schematic illustration of a connection between a vibrating device and a charging dock of the vibrating device, according to some embodiments of the invention

FIGS. 13A-13D are schematic illustrations of an additional assembly between a dental mirror and a vibrating device, according to some embodiments of the invention;

FIGS. 14A-14C are schematic illustrations of a vibrating device, according to some embodiments of the invention; and

FIG. 14D is a schematic longitudinal cross-section view of the assembly described in FIGS. 14A-14C, according to some embodiments of the invention.

### DESCRIPTION OF SPECIFIC EMBODIMENTS OF THE INVENTION

The present invention, in some embodiments thereof, relates to a vibrating device and, more particularly, but not exclusively, to a vibrating device for reducing dental pain.

An aspect of some embodiments relates to vibrating the inner side of a cheek and/or the inner side of a lip with sufficient amplitude to reduce pain in a different target tissue. In some embodiments, vibration of the inner side of the cheek with sufficient amplitude reduces sensitivity to pain at a different target tissue.

Alternatively, vibration of the inner side of the upper lip or the inner side of the lower lip with sufficient amplitude reduces sensitivity to pain at a different target tissue. In some embodiments, the inner side of a cheek and/or the inner side of a lip is vibrated with sufficient amplitude prior to a needle insertion into the periodontal tissue of a patient, for example when locally anesthetizing the periodontal tissue. In some embodiments, a sufficient vibration amplitude is a vibration amplitude that reduces pain at a desired target site or reduces sensitivity to pain at the desired target site. In some embodiments, the different target tissue is located at a distance of at least 1 centimeter (cm) from the vibrated site.

In some embodiments, the vibration amplitude is adjusted according to the distance from the target tissue. Alternatively or additionally, the vibration amplitude is adjusted according to the subject's age, gender and/or pain sensitivity. In some embodiments, a dentist receives an indication when the vibration reaches a desired vibration amplitude, for example a light indication and/or a sound indication.

An aspect of some embodiments relates to vibrating a dental mirror according to a vibration protocol. In some embodiments, the inner side of the cheek is vibrated according to a vibration protocol. Optionally, the dental mirror is vibrated according to a vibration protocol while contacting the inner side of a cheek and/or the inner side of a lip.

In some embodiments, the vibration protocol comprises at least one vibration parameter, for example vibration frequency, maximal vibration amplitude, overall vibration time period and/or vibration duration at maximal vibration amplitude. In some embodiments, the minimal vibration amplitude is at least 2 mm, for example 2 mm cm, 5 mm, 10 mm or any intermediate, smaller or larger value. In some embodiments, the minimal vibration amplitude is at least 2 cm, for example 3 cm, 4 cm, or 5 cm. In some embodiments, the vibration frequency is in a range of 2 Hz to 200 Hz, for example 2-50 Hz, 40-100 Hz or 80-200 Hz. In some embodiments, the vibration frequency is in a range of 10-12 Hz and/or in a range of 16-20 Hz or any intermediate, smaller or larger frequency. In some embodiments, the dental mirror vibrates in an axial movement and/or in a rotational movement. In some embodiments, the attachment piece, for example a dental mirror, placed in contact with the tissue delivers a force in a range of 5-55 gr to the tissue, for example 5-20 gr, 15-30 gr, 25-55 gr or any intermediate, smaller or larger force is delivered to the tissue by the attachment piece. In some embodiments, an indication is delivered when the at least one vibration parameter reaches a desired value. In some embodiments, an indication is delivered if the vibration protocol reaches a desired protocol stage.

An aspect of some embodiments relates to a dental mirror shaft comprising a mechanical interference element which resists vibrations of a vibrating device. In some embodiments, the dental mirror shaft comprises a first interlocking geometry that is shaped to mechanically interlock with a second interlocking geometry located in the vibrating device. In some embodiments, the first interlocking geometry comprises at least one recess, at least one window or at least one protrusion that mechanically interlocks with an interference mechanism which comprises the second interlocking geometry in the vibrating device. In some embodiments, the interference mechanism comprises an interference fit mechanism, for example a snap-fit mechanism. In some embodiments, the interference mechanism comprises a press fit mechanism, for example a press fit ball plunger. In some embodiments, the mechanical interference between the first interlocking geometry of the dental mirror shaft and the second interlocking geometry of the vibrating device resist rotational and/or axial movements of the shaft, for example movements that are caused by the vibration device.

According to some embodiments, at least part of the shaft has a non-circular cross section or an asymmetrical cross section. In some embodiments, the non-circular cross section or the asymmetrical cross section is shaped and sized to conform to an inner lumen of a vibrating device. In some embodiments, the non circular or the asymmetrical cross section of the shaft allows, for example, to prevent the rotation of the shaft inside the lumen of the vibrating device. Alternatively or additionally, the non circular or the asymmetrical cross section of the shaft allows, for example, inserting the shaft into the lumen of the vibrating device in a specific and optionally in a selected orientation between the shaft and the vibrating device. In some embodiments, the dental mirror shaft length is shorter than 18 cm, for example 15 cm, 12 cm, 10 cm or any intermediate, shorter or longer length.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Pain Reducing Effect

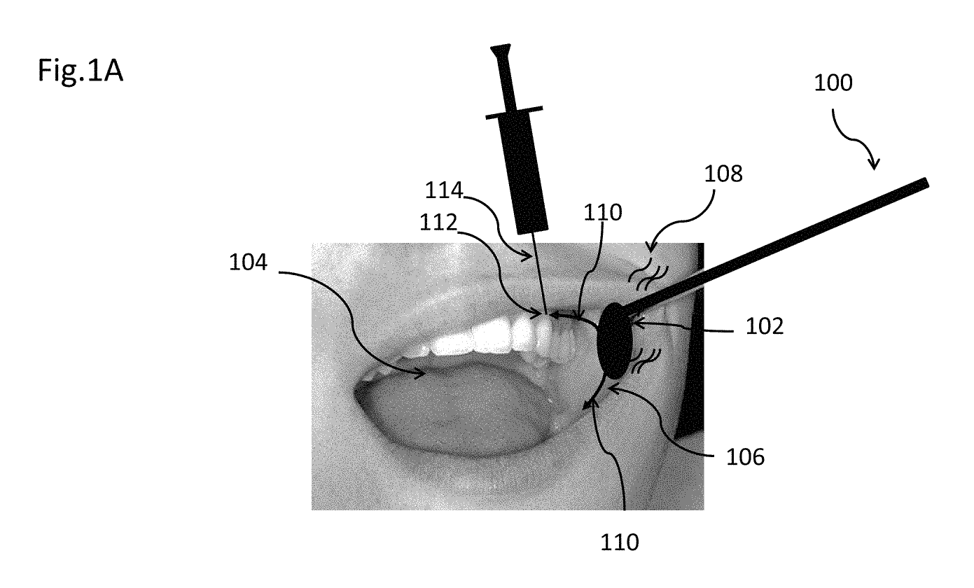

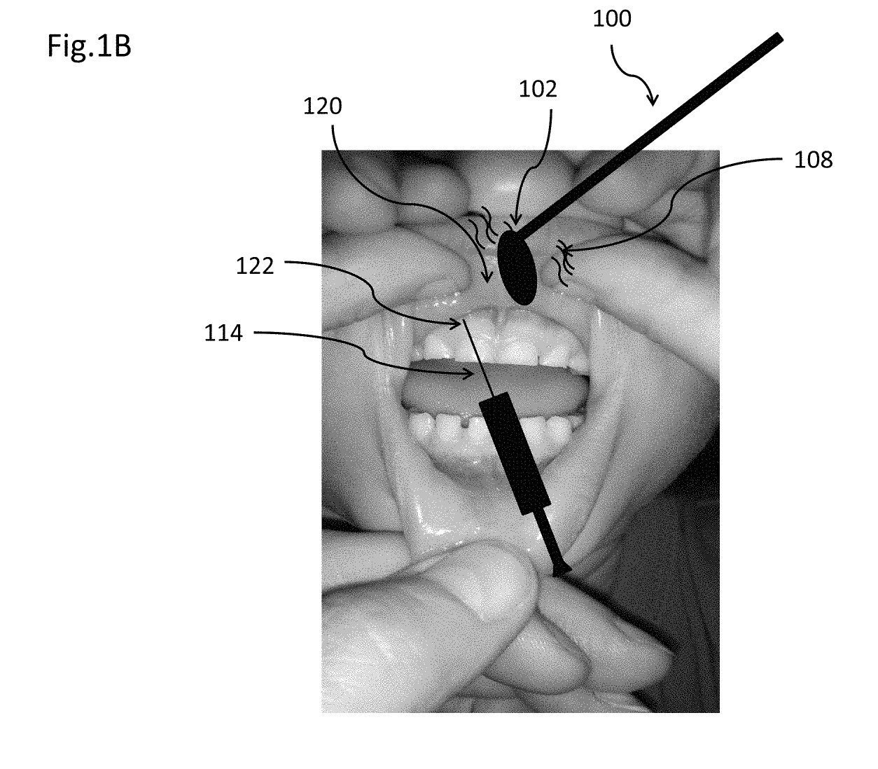

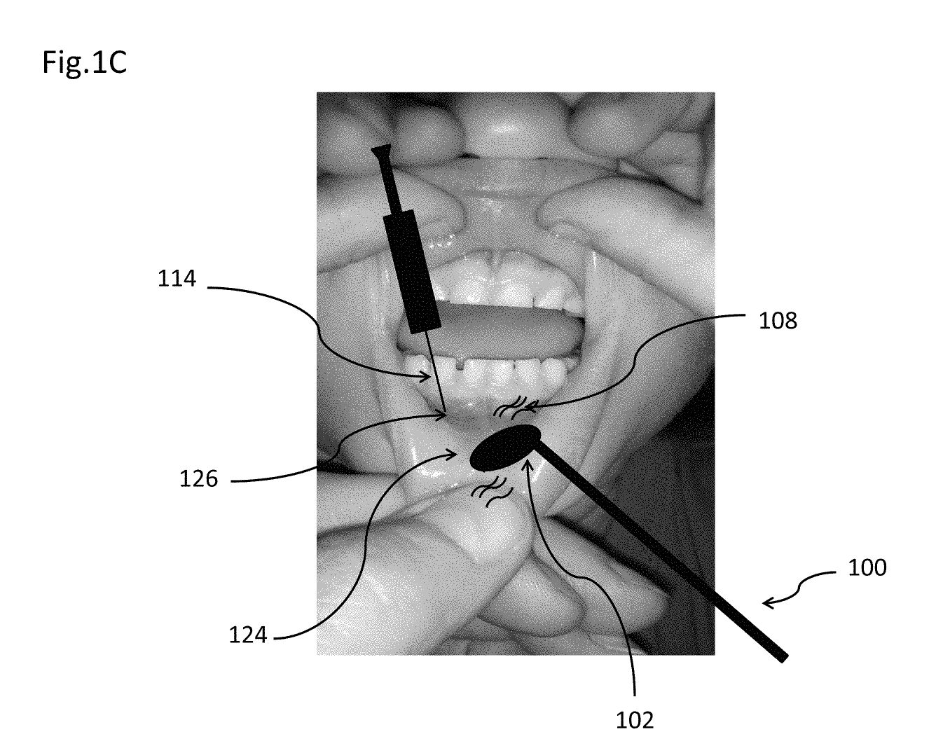

Reference is now made to FIGS. 1A-1C, depicting the effect of vibration on reducing pain sensitivity at a distant target site, according to some embodiments of the invention.

According to some exemplary embodiments, at least part of a dental device 100, for example a vibrating dental mirror 102 is inserted into the oral cavity 104 of a patient. In some embodiments, the vibrating dental mirror is placed in contact with the inner side 106 of a cheek or the inner side of a lip. Alternatively, the vibrating dental mirror contacts the inner side 106 of the cheek and part of the periodontal tissue.

Optionally, the dental mirror contacts the inner side 106 of the cheek or the inner side of a lip in an angle, for example to allow visualization of a treatment target site at the lower or upper periodontal tissue. In some embodiments, the dental mirror is then vibrates 108 with sufficient amplitude to remotely affect sensitivity to pain at a distant target site, for example target site 112. Without being bound to theory, activation of nerves which do not transmit pain signals, for example non nociceptive fibers 100 interfere with signals from pain fibers, thereby inhibiting pain.

According to some exemplary embodiments, the inner side 106 of the cheek or the inner side of a lip is vibrated prior to and/or during a periodontal treatment, for example insertion of needle 114 into the periodontal tissue at target site 112.

Optionally, vibration is applied after a periodontal treatment.

According to some exemplary embodiments, the inner side of the upper lip 120 is vibrated, for example as shown in FIG. 1B. In some embodiments, the inner side of the upper lip 120 is vibrated at a vibration site with sufficient amplitude to reduce sensitivity to pain at a different target site, for example target site 120. In some embodiments, the inner side of the upper lip is vibrated prior or during the insertion of a needle 114 into the periodontal tissue or at a target site between the periodontal tissue and the inner side of the upper lip. Alternatively, the inner side of the lower lip 124 is vibrated at a vibration site with sufficient amplitude, to reduce sensitivity to pain at a different target site, for example target site 126. In some embodiments, the distance between the vibration site and the target site, for example target sites 112, 122 or 126 is at least 1 cm, for example 2, 3, 5, 6, 7 cm.

### Exemplary Pain Reducing Process

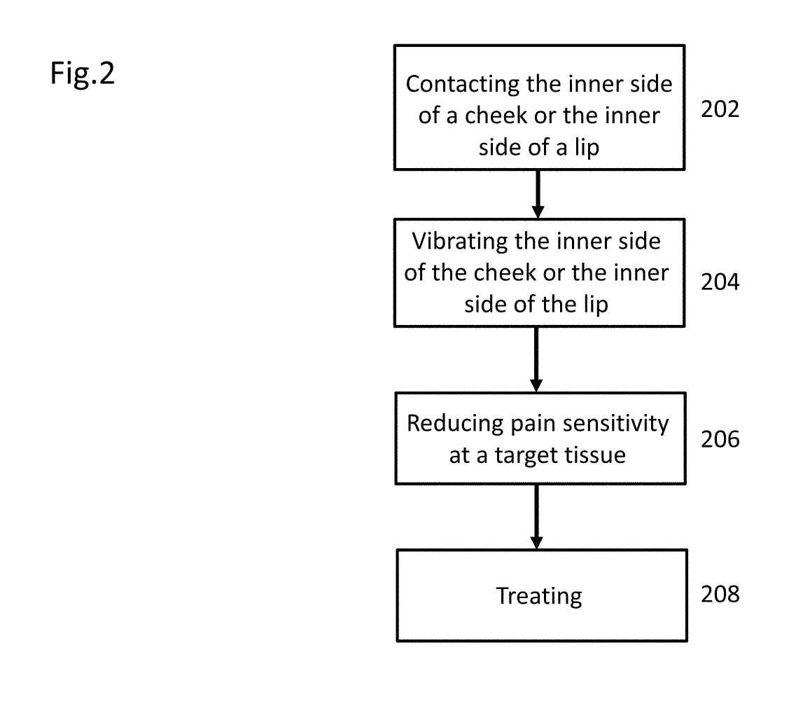

Reference is now made to FIG. 2, depicting a pain reducing process according to some embodiments of the invention.

According to some exemplary embodiments, at least part of a vibrating device, for example a vibrating dental mirror is placed in contact with the inner side of a cheek and/or with the inner side of a lip at 202. In some embodiments, the vibrating dental mirror is placed in contact while stretching the inner side of the cheek and/or the inner side of the lip. In some embodiments, the vibrating dental mirror contacts the inner side of the cheek or the inner side of a lip at a selected contact site. In some embodiments, the contact site is selected based on the distance to a desired target site.

Alternatively or additionally, the contact site is selected based on the age of the patient and/or the gender of the patient. Optionally, the contact site is selected based on the patient's sensitivity to pain, for example if the patient is highly sensitive to pain than the dental mirror will be placed near the desired target site. In some embodiments, the contact site is selected based on the proximity of the contact site to neural pathways, for example neural pathways that lead to the desired target site.

According to some exemplary embodiments, the contact site is located at a distance of at least 1 cm, for example 1 cm, 2 cm, 3 cm, 5 cm or any intermediate, smaller or larger distance from the desired target site.

According to some exemplary embodiments, the vibrating dental mirror delivers vibrations to the inner side of the cheek and/or to the inner side of a lip at 204. In some embodiments, the vibrating dental mirror vibrates the inner side of the cheek and/or the inner side of the lip according to a vibration protocol. In some embodiments, the vibration protocol comprises vibration frequency, vibration amplitude, and/or vibration duration. In some embodiments, the vibrating dental mirror vibrates the inner side of the cheek and/or the inner side of a lip with sufficient amplitude to affect sensitivity to pain and/or to reduce pain at distant target sites, for example target sites located at the periodontal tissue. In some embodiments, the vibrating dental mirror vibrates the inner side of the cheek and/or the inner side of a lip with a maximal vibration amplitude of at least 2 cm, for example 3 cm, 4 cm, or 5 cm. In some embodiments, the vibrating dental mirror vibrates the inner side of the cheek with a vibration frequency in a range of 2 Hz to 200 Hz, for example 2-50 Hz, 40-100 Hz or 80-200 Hz. In some embodiments, the vibrating dental mirror vibrates the inner side of a cheek with a vibration frequency of 10-12 Hz, 12-16 Hz, or 16-20 Hz or any intermediate, smaller or larger frequency.

According to some exemplary embodiments, the vibrating dental mirror is vibrated in circular movements. Alternatively, the vibrating dental mirror vibrates in axial movements. Optionally, the vibrating dental mirror vibrates in both axial and circular movements.

According to some exemplary embodiments, following vibration of the inner side of the cheek and/or the inner side of a lip, sensitivity to pain is reduced at the target site at 206. Without being bound to theory, vibration of the inner side of the cheek and/or the inner side of a lip stimulates large diameter fibers that interfere with pain-related signals delivered by thin diameter nerve fibers at the distant target site.

Alternatively or additionally and without being bound by theory, vibration of the inner side of the cheek prevents or reduces the ability to generate pain-relates signals at the target site. In some embodiments, vibration of the inner side of the cheek and/or the inner side of a lip reduces pain and/or reduces sensitivity to pain at the target site.

According to some exemplary embodiments, following or during pain sensitivity reduction, the target site is treated at 208. In some embodiments, a needle is inserted into the periodontal tissue at the target site following or during pain sensitivity reduction, for example during the application of a local anesthetic drug.

### Exemplary Changes in Vibration Amplitude Level

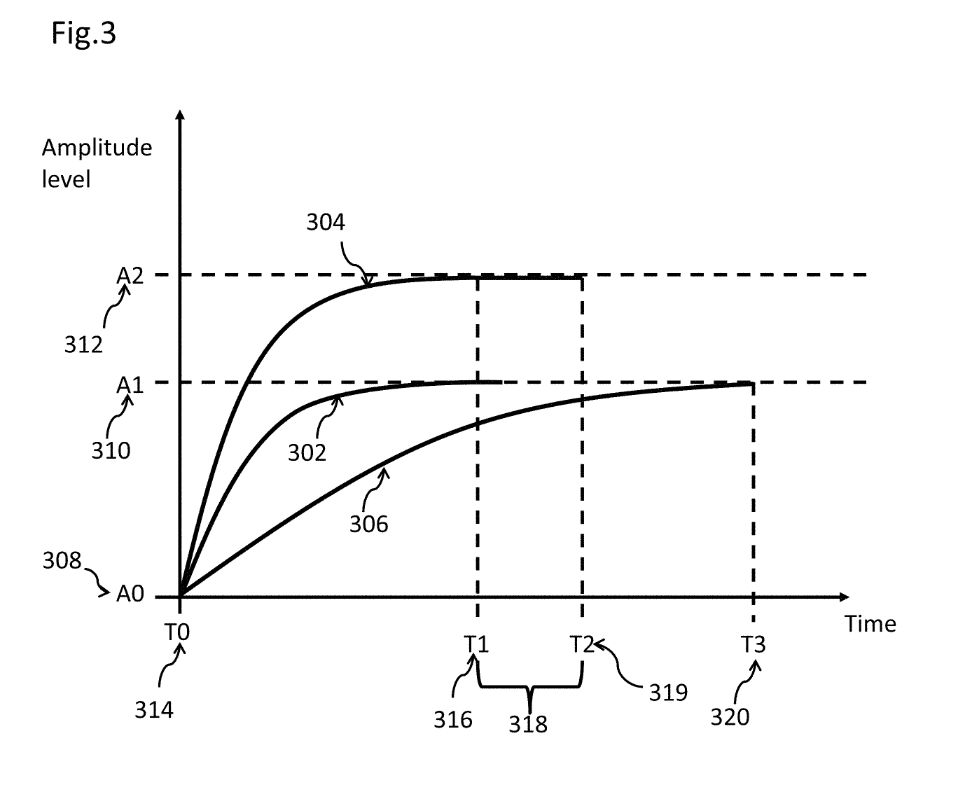

Reference is now made to FIG. 3 depicting changes in vibration amplitude over time, according to some embodiments of the invention.

According to some exemplary embodiments, the dental vibration device vibrates the inner side of the cheek according to a vibration protocol. In some embodiments, the vibration protocol comprises overall vibration duration, vibration frequency, maximal vibration amplitude level, vibrating time at maximal amplitude and time to reach maximal amplitude.

According to some exemplary embodiments, when the dental device vibrates the dental mirror, the vibration amplitude changes from a zero amplitude to a maximal amplitude level, for example from zero amplitude A0 308 to desired amplitude A1 310, as shown in graph 302. In some embodiments, the maximal amplitude level is selected based on the distance from the target site. Alternatively, the maximal amplitude level is selected based on the patient's gender and/or age.

Optionally, the maximal amplitude level is selected based on the patient's sensitivity to pain.

In some embodiments, a higher vibration amplitude level is necessary, for example in order to reach the desired pain reduction effect. In some embodiments, for example as shown in graph 304, a higher vibration amplitude level is selected, for example vibration amplitude 312. Optionally, the time needed to reach the maximal amplitude level in different vibration protocols is similar. In some embodiments, the time T1 316 needed to reach amplitude level 310 is similar to the time needed to reach amplitude level 312.

According to some exemplary embodiments, when the maximal vibration amplitude in a specific vibration protocol is reached, the maximal vibration amplitude is applied for a pre-determined time, for example time period 318 between T2 319 and T1 316. Alternatively, the maximal vibration amplitude is applied until a user of the vibrating device, for example a dentist, stops the vibration.

According to some exemplary embodiments, the time or duration needed to reach the maximal vibration amplitude is changed. In some embodiments, for example as shown in graph 306, the time T3 320 needed to reach vibration amplitude A1 is longer compared to time T1 316 in graph 302.

### Exemplary Dental Vibration Device

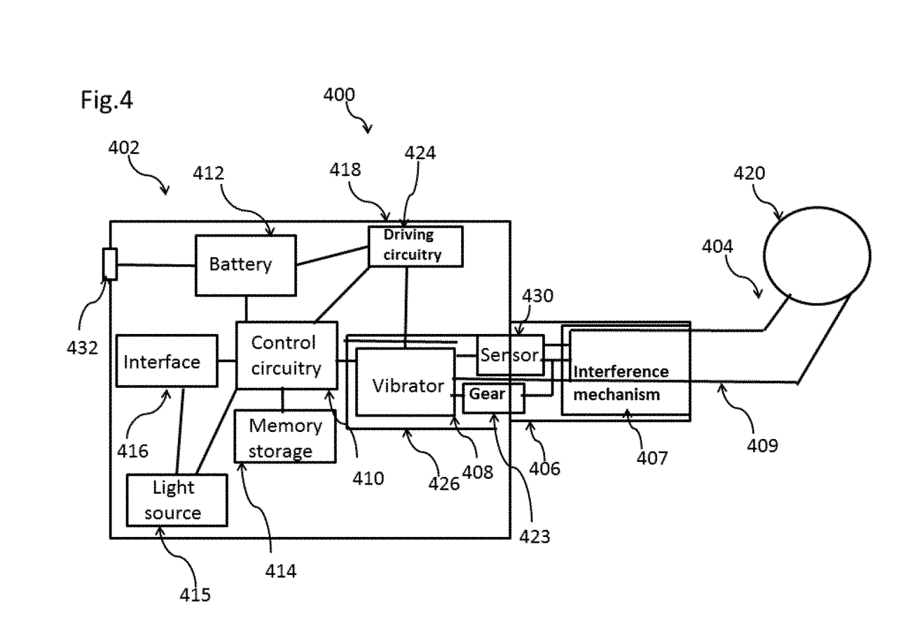

Reference is now made to FIG. 4 depicting components of a dental vibration device, according to some embodiments of the invention.

According to some exemplary embodiments, dental vibration device, for example device 400 comprises a handle 402 and an attachment piece 404. As used herein, in some embodiments the attachment piece is a dental mirror assembly comprising a dental mirror mechanically connected to a handle or to a shaft. In some embodiments, the dental mirror and the handle, or the dental mirror and the shaft are manufactured as an inseparable single unit. Alternatively, the dental mirror is a connectable dental mirror that can be connected to the handle or to the shaft via a connector. In some embodiments, the dental mirror is connected to the handle or to the shaft by rotation or by an interference mechanism or an interlocking mechanism.

According to some embodiments the attachment piece 404 is a reusable attachment piece, optionally configured to be sterilized, for example by ultraviolet (UV) radiation. Alternatively, the attachment piece 404 is a single use attachment piece. In some embodiments, handle 402 comprises casing 418 which is shaped and sized to be handheld by a user of the device, for example a dentist. In some embodiments, attachment piece 404 comprises a dental mirror 420 or any geometrical structure which is shaped and sized for contacting a deformable tissue of the mouth, for example the inner side of a cheek and/or the inner side of a lip. In some embodiments, the dental mirror 420 or the geometrical structure is round and has a diameter in a range of 0.5-5 cm, for example 1 cm, 2 cm, 3 cm or any intermediate, smaller or larger value.

According to some exemplary embodiments, the attachment piece 404 is connected to a vibrator 408 placed within handle 402. In some embodiments, the vibrator comprises a motor, for example an electric motor. In some embodiments, the attachment piece 404 is connected to the vibrator 408 through a gear 423. In some embodiments, a damper 426 is positioned around vibrator 408, and serves to reduce the effect of vibrations generated by the vibrator 408 on handle 402. Alternatively or additionally, a damper is positioned around handle 402, for example to reduce the delivery of vibrations generated by the vibrating device 400 to a hand of a user holding the device. In some embodiments, the damper comprises an elastic material, for example gel or rubber. In some embodiments, vibrator 408 vibrates attachment piece 404, under the control of control circuitry 410.

According to some embodiments, the force delivered by the dental mirror 420 to the tissue, caused by the vibrations of the vibrator, is in a range of 5-55 gr to the tissue, for example 5-20 gr, 15-30 gr, 25-55 gr or any intermediate, smaller or larger force is delivered to the tissue by the attachment piece. In some embodiments, the vibrator generates a maximal energy of 1 watt, for example 0.8 watt, 0.5 watt, 0.3 watt or any intermediate smaller or larger value.

In some embodiments, at least one sensor 430 is coupled between vibrator 408 and attachment piece 404. In some embodiments, sensor 430 senses the vibration amplitude of the attachment piece 404 and optionally also the vibration amplitude generated by vibrator 408. In some embodiments, sensor 430 delivers the measured values to a control circuitry 410.

In some embodiments, control circuitry 410 determines whether the measured vibration amplitude has reached a desired value, based on the measured values.

Alternatively or additionally, control circuitry determines the difference between the vibration amplitude of the attachment piece 404 compared to the vibration amplitude generated by vibrator 408. In some embodiments, if the vibration amplitude of the attachment piece 404 is lower in more than 33% or 44% or 50% compared to the vibration amplitude generated by the vibrator 408, then control circuitry 410 signals interface 416 to generate a human detectable alert signal.

According to some exemplary embodiments, the interface 416 comprises at least one selector and/or at least one button for example for activating the vibrating device, selecting a vibration frequency and/or vibration time and/or for activating at least one light source located in the vibrating device.

According to some exemplary embodiments, the vibration device 400, comprises at least one light source 415, for example a LED light source for lighting the dental mirror and/or a region next to the dental mirror. In some embodiments, the at least one light source illuminates the contact site of the attachment piece or the dental mirror with the deformable tissue of the mouth, for example the inner side of a cheek and/or the inner side of a lip. Alternatively or additionally, the at least one light source illuminates the desired target site. In some embodiments, the at least one light source is activated and/or deactivated independently from the vibrator activation.

Alternatively, the at least one light source is activated automatically when the vibrator is activated. In some embodiments, the at least one light source emits light directed to the dental mirror. Alternatively or additionally, the at least one light source emits light directed to a selected syringe insertion region and/or to a selected dental treatment region. In some embodiments, the at least one light source is electrically connected to the control circuitry 410 and/or to interface 416.

According to some exemplary embodiments, the control circuitry 410 controls the vibration of vibrator 408. Optionally, the control circuitry 410 controls the vibrations of vibrator 408 by controlling driving circuitry 424. In some embodiments, driving circuitry 424 controls the electric power delivery from battery 412 to the vibrator 408. In some embodiments, control circuitry 410 signals vibrator 408 to generate vibrations, according to at least one vibration protocol stored in memory 414. In some embodiments, the vibration protocol comprises the overall duration of the vibration, the vibration frequency, the vibration maximal amplitude and/or the vibration time at maximal amplitude. Optionally, sensor 430 measures vibration frequency of the attachment piece 404.

According to some exemplary embodiments, the memory 414 stores values of at least one vibration parameter or indications thereof. In some embodiments, the memory 414 stores at least one vibration protocol or indications thereof. In some embodiments, the memory 414 stores at least one table and/or at least one algorithm for adjusting values of at least one vibration parameter or at least one parameter of a vibration protocol based on the age of a patient and/or clinical condition of the patient.

According to some exemplary embodiments, vibrator 408 vibrates the attachment piece 404, for example a handle or a shaft connected to a dental mirror in a frequency range of 10-12 Hz, 12-16 Hz, or 16-20 Hz or any intermediate, smaller or larger frequency. In some embodiments, the vibrator 408 rotates the dental mirror 420 around the attachment piece axis, for example the handle axis in a repetitive angular movement of up to 35 degrees, for example 35 degrees, 30 degrees, 25 degrees, 22 degrees or any intermediate, smaller or larger angular movement

According to some exemplary embodiments, battery 412 is a rechargeable battery, for example a lithium ion battery. In some embodiments, battery 412 is charged by connection of an external charger to charging connector 432, placed in handle 402. Alternatively or additionally, battery 412 is a replaceable battery.

According to some exemplary embodiments, attachment piece 404 is connected to handle 402 via adaptor 406. In some embodiments, adaptor 406 allows, for example easy removal and connection of attachment piece 404 to handle 402. In some embodiments, adaptor 406 comprises a locking mechanism to allow, for example to lock the attachment piece 404 to handle 402 after connection. In some embodiments, adaptor 406 is a reusable adaptor configured to be sterilized, for example by UV radiation. Alternatively, the adaptor is a single use adaptor.

According to some exemplary embodiments, the device 402 comprises a an interference mechanism 407 within handle 402 that mechanically interferes with an interlocking geometry comprising a recess, an opening, a window or a protrusion in attachment piece handle 409 or in a shaft connected to mirror 420. In some embodiments the interference mechanism 407 comprises a rubber the mechanically interferes with the attachment piece handle 409 or the shaft by generating sufficient friction that at least partly fixes the attachment piece handle inside the handle 402. Alternatively, the interference mechanism 407 comprises a button or a screw that interferes with the attachment piece handle 409 or the shaft by application of force from the side. In some embodiments, in order to release the attachment piece handle 409 or the shaft from the handle 402 of the device 400, a button or a selector is presses, or moved or turned.

According to some exemplary embodiments, device 400 comprises a gear 423 mechanically coupled between vibrator 408 and the attachment piece handle 409 or the shaft of the attachment piece. In some embodiments, gear 423 comprises an eccentric transmission or a reciprocating transmission. In some embodiments, the gear 423 mechanically delivers the vibrations generated by the vibrator 408 to the attachment piece. In some embodiments, the gear 423 is under the control of control circuitry 410, for example to adjust at least one vibration parameter, for example the vibration frequency and/or vibrations amplitude and/or the vibration power, delivered to the attachment piece. Optionally, the control circuitry adjusts the at least one vibration parameter based on at least one vibration parameter indication stored in memory storage 414.

According to some exemplary embodiments, the gear 423 is a reciprocating transmission which transforms rotational movement of the vibrator 408 to a reciprocating movement of the attachment piece 404. In some embodiments, the gear 423 transforms a rotational movement of the vibrator 408, to a reciprocating movement of the dental mirror 420. In some embodiments, the gear 423 transforms a rotational movement of the vibrator 408 to a reciprocating sideways motion of the dental mirror 420 relative to the handle 402. In some embodiments, the reciprocating motion is a reciprocating motion of at least 5 degrees to each side, for example 10 degrees, 15 degrees, 20 degrees, 25 degrees or any intermediate, smaller or larger value.

According to some exemplary embodiments, the gear 423 is a back-drive gear. In some embodiments, the back-drive gear reduces the power generated by the vibrator, for example when the vibration amplitude of the dental mirror is reduced. Alternatively or additionally, the back-drive gear reduces the power generated by the vibrator, when an external force is applied on the vibrating attachment piece, for example by a tissue which is in contact with the vibrating dental mirror.

According to some exemplary embodiments, the weight of the attachment piece 404 is in a range of 5-20 gr, for example 6 gr, 10 gr, 12 gr, 12.5, 15 gr or any intermediate, smaller or larger value. In some embodiments, the weight of the handle 402, is in a range of 100-200 gr, for example 120 gr, 150 gr, 170 gr or any intermediate, smaller or larger value. In some embodiments, the weight ratio between the handle 402 and the attachment piece 404 is in a range of 1:7 to 1:15, for example 1:10, 1:11, 1:12 or any intermediate, smaller or larger weight ration.

### Exemplary Vibration Process

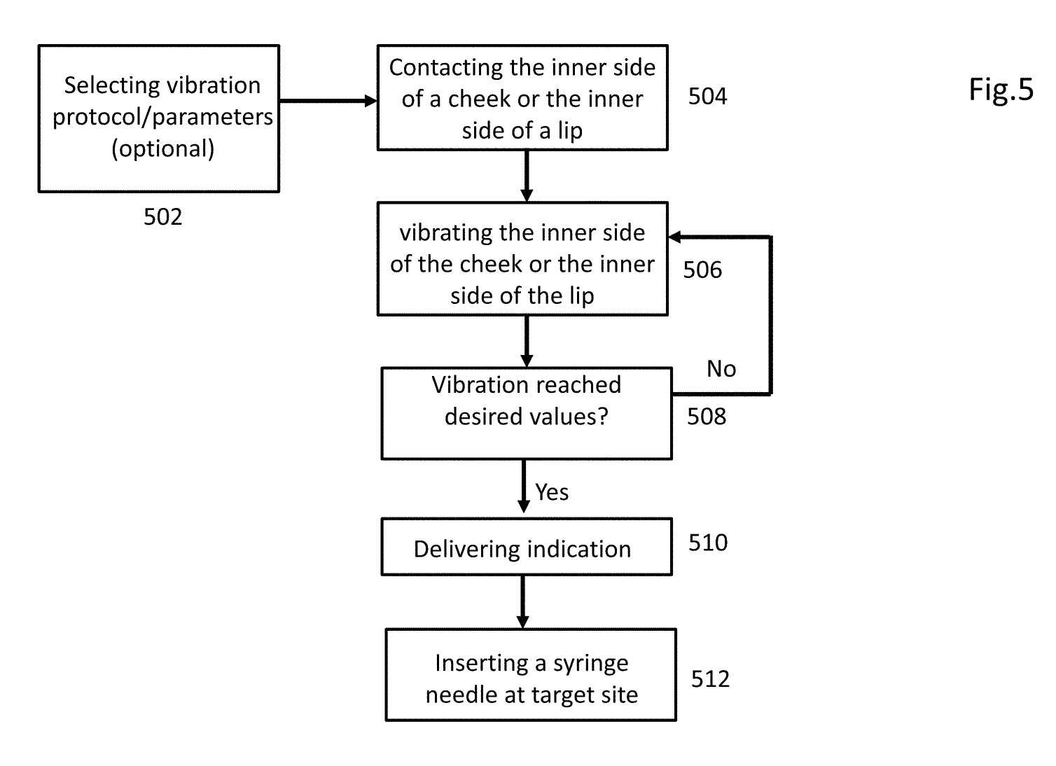

Reference is now made to FIG. 5, depicting a vibration process, according to some embodiments of the invention.

According to some exemplary embodiments, a vibration protocol is selected at 502. Alternatively, vibration parameters are selected at 502. In some embodiments, the vibration parameters comprise vibration duration, vibration frequency, maximal vibration amplitude and/or vibration time at maximal vibration amplitude. In some embodiments, the vibration protocol and/or vibration parameters are fixed.

According to some exemplary embodiments, at least part of the vibrating device, for example a vibrating dental mirror, is placed within the mouth of a patient, in contact with the inner side of a cheek and/or the inner side of a lip at 504. In some embodiments, the contact site is selected based on the distance from a desired pain reduction target site. Alternatively or additionally, the contact site is selected based on at least one vibration parameter.

According to some exemplary embodiments, the vibrating dental mirror vibrates the inner side of the cheek at 506. In some embodiments, the inner side of the cheek is vibrating according to the selected vibration protocol and/or according to the selected vibration parameters.

According to some exemplary embodiments, the vibration parameters are monitored during the vibration process, for example in order to decide whether the vibration reached the desired parameter values at 508. Alternatively or additionally, the vibration protocol progression is monitored, for example to determine whether the vibration protocol is completed. In some embodiments, if the vibration did not reach desired parameter values and/or that the vibration protocol is not finished, then the vibrating device continues to vibrate the inner side of the cheek at 506.

According to some exemplary embodiments, if the vibration protocol reaches a desired protocol stage and/or if the vibration reaches a desired parameter value then an indication is delivered at 510. In some embodiments, the indication is a human detectable indication, for example a light indication and/or a sound indication. In some embodiments, if at least one measured parameter value is different from a desired value, a human detectable alert indication is provided.

According to some exemplary embodiments, during the vibration of the inner side of the cheek or after the vibration is completed, a syringe needle is inserted at the target site, for example into the periodontal tissue at the target site at 212.

### Exemplary Vibrating Dental Mirror

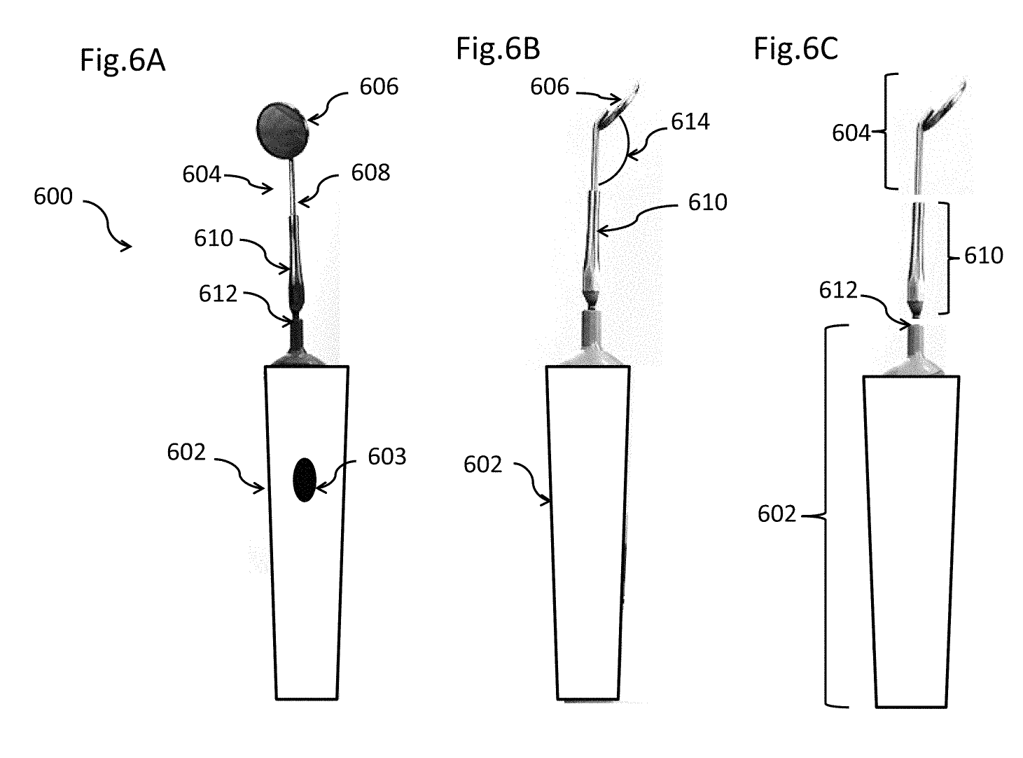

Reference is now made to FIGS. 6A-6C, depicting a vibrating dental mirror device, according to some embodiments of the invention.

According to some exemplary embodiments, a vibrating device for example vibrating device 600 comprises a handle 602 and a vibrating attachment piece, for example a vibrating dental mirror 604. In some embodiments, the vibrating dental mirror 604 comprises an elongated shaft 608 with a dental mirror 606 connected to the distal end of shaft 604.

In some embodiments, the vibrating dental mirror 604 is connected to handle 602 via connector 612. In some embodiments, connector 612 allows for example, easy connection and removal of the vibrating attachment piece. Optionally, connector 612 comprises a locking mechanism, for example to allow locking of the attachment piece to the handle.

In some embodiments, the vibrating dental mirror 604 is connected to the handle via an adaptor 610. In some embodiments, adaptor 610 is an elongated shaft.

In some embodiments adaptor 610 comprises a first proximal connection member, for example to allow connection to handle 602, and a second distal connection member, for example to allow connection to the vibrating dental mirror 604.

In some embodiments, the dental mirror 606 is positioned in an angle 614 to shaft 608 or to the handle 602. In some embodiments, positioning the dental mirror in an angle 614 allows better access to the inner side of the cheek and/or better visualization of the target site during vibration.

In some embodiments, handle 602 comprises a selector 603, optionally connected to interface 416 of FIG. 4. In some embodiments, selector 603 is used to activate and/or to deactivate vibrating device 600. Alternatively or additionally, selector 603 is used to select a vibration protocol and/or at least one vibration parameter.

### Exemplary Vibrating Dental Mirror Inside a Patient's Mouth

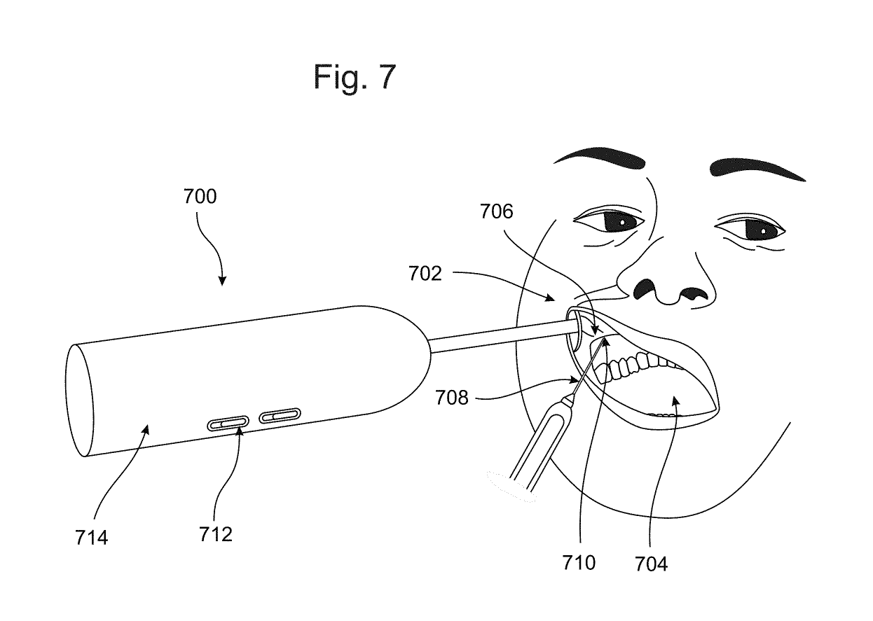

Reference is now made to FIG. 7, depicting a vibrating dental mirror inside the mouth of a patient, according to some embodiments of the invention.

According to some exemplary embodiments, a vibrating device 700 comprising a vibrating dental mirror 702 is placed at least partly inside a mouth 704 of a patient. In some embodiments, the vibrating dental mirror is placed in contact with the inner side 706 of a cheek. Alternatively, the vibrating dental mirror is placed in contact with the inner side of a lip. In some embodiments, the vibrating dental mirror 702 vibrates the inner side of the cheek at a vibration site with sufficient amplitude for example, to reduce sensitivity to pain at a different target site, for example target site 710. In some embodiments, the target site is in the basis of the periodontal tissue or in the interface between the periodontal tissue and the inner surface of the lip or the cheek. In some embodiments, the inner side of the cheek is vibrated prior to and/or during the insertion of a needle 708 into the periodontal tissue at target site 710.

In some embodiments, the vibrating device is activated using at least one controller button 712 positioned on the handle 714 of the vibrating device 700.

Optionally, the at least one controller button, for example controller button 712 is used for selection of a desired vibration protocol. In some embodiments, the distance between the vibration site and the target site, for example target site 710 is at least 1 cm, for example 2, 3, 5, 6, 7 cm.

### Exemplary Connections between a Dental Mirror and a Vibrating Device

According to some exemplary embodiments, the vibrating device is connected to dental mirrors or to dental mirrors with handles. In some embodiments, the vibrating device includes an internal handle, which optionally extends from the vibrating device, for example to allow connection between a dental mirror and the vibrating device. In some embodiments, the vibrating device is shaped and sized, for example to allow connection to commercially available dental mirrors with or without handles of different manufacturers. Alternatively, the vibrating device is shaped and sized, for example to allow connection of specialized dental mirrors and/or specialized handles, optionally through a specific geometrical connector.

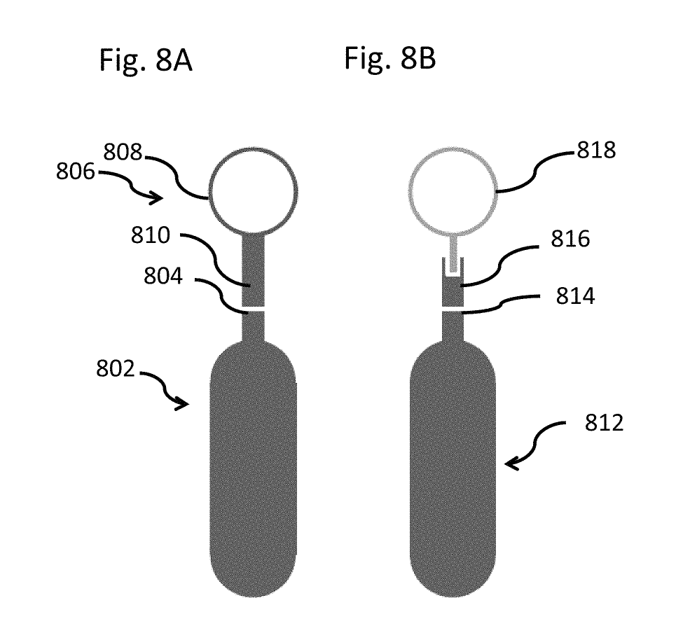

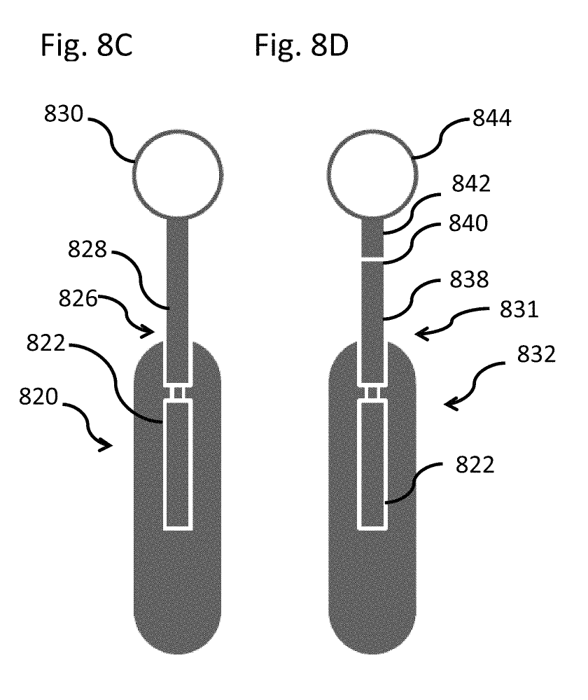

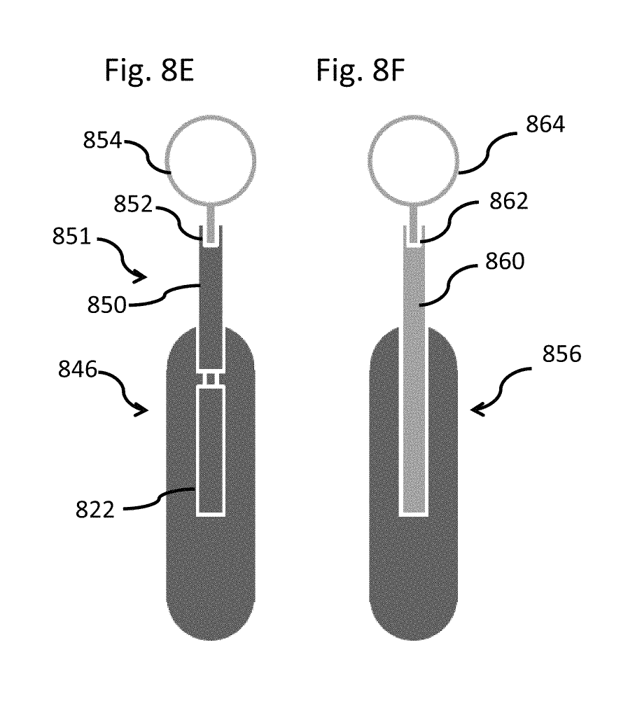

Reference is now made to FIGS. 8A-8F depicting different connections between a dental mirror and a vibrating device, according to some exemplary embodiments of the invention.

According to some exemplary embodiments, for example as shown in FIG. 8A, a dental mirror assembly, for example dental mirror assembly 806 is connected to a vibrating device, for example vibrating device 802 via a coupling element, for example connector 803 which extends from the vibrating device 802 body. In some embodiments, an axial shaft or a handle, for example handle 810 of the dental mirror is connected to the connector 803 of the vibrating device 802.

According to some exemplary embodiments, for example as shown in FIG. 8B a vibrating device, for example vibrating device 81 comprises a coupling element 814 shaped and sized, for example to allow coupling to an adaptor, for example adaptor 816. In some embodiments, the coupling element 814 includes a geometrical, optionally an asymmetrical geometrical connector that optionally fits a complementary element in the adaptor 816. Additionally, the adaptor 816 includes a connector to the dental mirror 818. In some embodiments, the connector of the adaptor 816 is a universal connector that is shaped and sized, for example to allow connection of commercially available dental mirror of some manufacturers to the adaptor. In some embodiments, the adaptor has a universal distal end connector shaped and sized to allow connection to commercially available dental mirrors, and a unique proximal end connector, optionally a geometrical connector that allows, for example connection to the vibrating device, for example vibrating device 812.

According to some exemplary embodiments, for example as shown in FIG. 8C, a vibrating device, for example vibrating device 820 is shaped and sized to be connected to a shaft, for example shaft 826 of a dental mirror 830. In some embodiments, the shaft 826 comprises a section 828 which optionally has a rotational symmetry, and section 822 which has a non-circular cross-section 822. In some embodiments, the non-circular cross-section is shaped and sized to fit a mechanical joint and/or a mechanical interference lock inside the vibrating device, for example vibrating device 820.

According to some exemplary embodiments, for example as shown in FIG. 8D, a section of the shaft extending from the vibrating device, for example section 838 that optionally has a rotational symmetry comprises a coupling element, for example a connector 840 at the distal end of the shaft. In some embodiments, a distal end of a shaft is for example the shaft end that is positioned at the largest distance from the vibrating device. In some embodiments, the connector 840 is shaped and sized to allow connection of a dental mirror 844, optionally via extension 840 of the dental mirror 844 to the shaft 831. In some embodiments, proximal section 822 of the shaft 831 connects the shaft to the vibrating device, for example vibrating device 832. In some embodiments, a proximal section of a shaft is a section which is located at the largest distance from the dental mirror.

According to some exemplary embodiments, for example as shown in FIG. 8E, a distal section of the shaft extending from the vibrating device, for example distal section 850 comprises a universal connector 852 at the distal end of the shaft. In some embodiments, the universal connector 852 allows, for example connection of a commercially available dental mirror of at least some manufacturers, for example dental mirror 854 to the shaft 851. In some embodiments, proximal section 822 of the shaft connects the shaft to the vibrating device, for example vibrating device 846.

According to some exemplary embodiments, for example as shown in FIG. 8F, the vibrating device, for example vibrating device 856 is shaped and sized to allow connection of at least some commercially available handles, for example handle 860. In some embodiments, handle 860 comprises a universal connector 862, that allows, for example to connect at least some commercially available dental mirrors, for example mirror 864 to the handle 860. In some embodiments, the device 856 comprises an interlocking geometry, for example an interlocking geometry of an interference mechanism that interlocks the handle 860 inside the vibrating device 856. In some embodiments, the interference mechanism mechanically interferes with the handle 860. In some embodiments, the interference mechanism comprises a rubber element within the vibrating device that optionally generates friction against the handle 860. Alternatively or additionally, the interference mechanism comprises a screw or a lever that applies a side force against the handle 860.

### Exemplary Dental Mirror and Handle

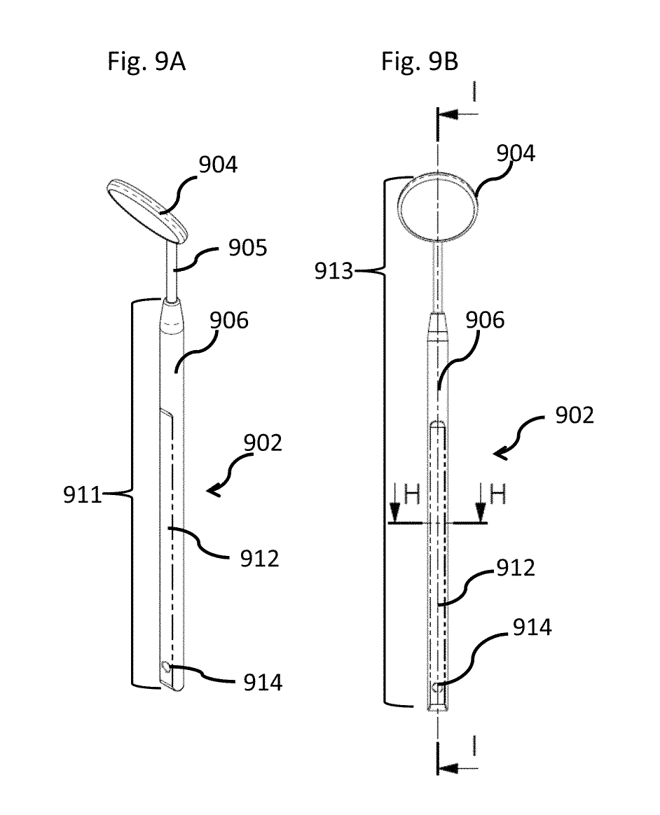

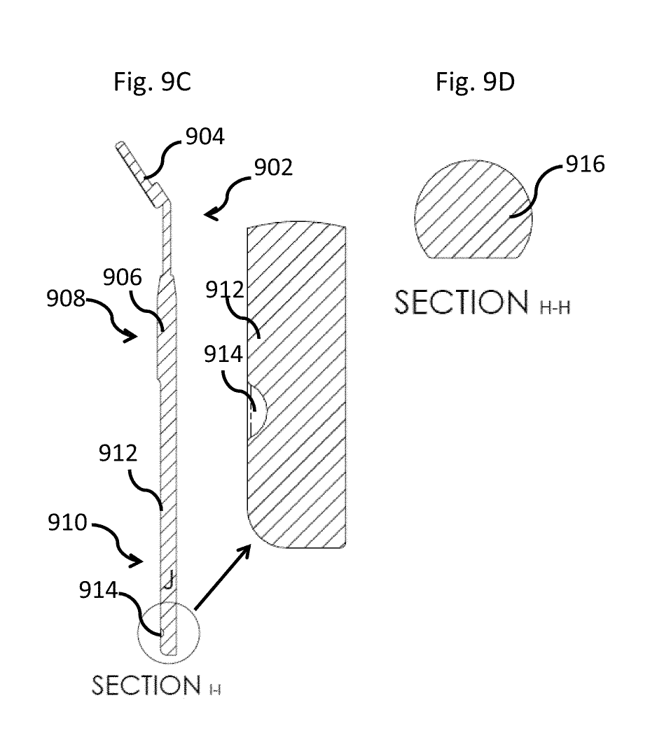

Reference is now made to FIGS. 9A-9D depicting a shaft and a mirror assembly, according to some exemplary embodiments of the invention.

According to some exemplary embodiments, a dental mirror, for example dental mirror 904 is connected to a shaft, optionally a short shaft, for example shaft 902. In some embodiments, shaft 902 has a distal section, for example distal section 906 located close to the dental mirror 904, and a proximal section, for example proximal section 912 located at a distance and away from the dental mirror 904. In some embodiments, at least part of the distal section 906 has a rotational symmetry. Additionally or alternatively, at least part of the distal section 906 has a symmetrical cross-section. In some embodiments, the diameter of the distal section 906 is in a range of 3 to 10 mm, for example 3 mm, 4 mm, 6 mm or any intermediate, smaller or larger value. In some embodiments, the diameter of an extension 905 connecting dental mirror 904 to shaft 902 is in a range of 1-4 mm, for example 2 mm, 2.5 mm, 3 mm or any intermediate, smaller or larger value. In some embodiments, the diameter of the mirror 904 is in a range of 0.5-4 cm, for example 1 cm, 2 cm, 3 cm or any intermediate, smaller or larger value.

According to some exemplary embodiments, the axial length 911 of the shaft 902 is up to 25 cm, for example 20 cm, 17 cm, 15 cm, 14 cm, 13 cm 12 cm, 11 cm, 10 cm or any intermediate, smaller or larger value. In some embodiments, the axial length 913 of an assembly comprising the dental mirror 904 or any tissue contacting piece and the shaft 902 is at least 10 cm, for example 14 cm, 16 cm, 18 cm, 20 cm or any intermediate, smaller or larger value.

According to some exemplary embodiments, at least part of the proximal section 912 has a non-circular cross-section. Additionally or alternatively, at least part of the proximal section 912 has an asymmetrical cross-section. In some embodiments, the proximal section 912 and/or the proximal section that has a non-circular cross-section is shaped and sized to conform to an inner lumen of a vibrating device, for example handle 402 shown in FIG. 4.

According to some exemplary embodiments, the shaft 902, optionally in the proximal section 912, comprises a first interlocking geometry, for example at least one opening, at least one window, at least one protrusion, or at least one recess, for example recess 914 that is shaped and sized for mechanically interlocking with a second interlocking geometry in a vibrating device, for example handle 402 shown in FIG. 4. In some embodiments, the second interlocking mechanism is part of the interference mechanism shown in FIG. 4. In some embodiments, recess 914 is shaped and sized to interlock with an interlocking geometry in vibrating device by interference fit, for example snap-fit, press-fit or any other mechanical interference mechanism. In some embodiments, the recess 914 is shaped and sized to fit a press-fit plunger, for example a ball-fit plunger or a spring-fit plunger in the vibrating device. In some embodiments, the recess 914 fits a mechanical joint in the vibrating device. In some embodiments, the interlocking between the shaft and the vibrating device prevents the release of the shaft from the vibrating device during vibration. Alternatively or additionally, the interlocking between the shaft and the vibrating device resist rotational movements of the shaft. In some embodiments, to release the shaft 902, a button in the vibrating device is pressed or a screw or a lever in the vibrating device is turned.

According to some exemplary embodiments, for example as shown in FIG. 9D, the cross-section 916 of at least part of the proximal section 912 is asymmetrical and/or is a non-circular cross-section. Optionally, the cross-section 916 is a D-shaped cross-section. In some embodiments, the asymmetrical and/or non-circular cross-section of the proximal section allows, for example, to insert the handle into the vibrating device in a selected orientation between the handle and the vibrating device. Alternatively or additionally, the asymmetrical and/or non-circular cross-section of the proximal section prevents, for example, relative movement, optionally relative rotational movement, between the handle and the vibrating device.

### Exemplary Coupling between a Vibrating Device and a Dental Mirror

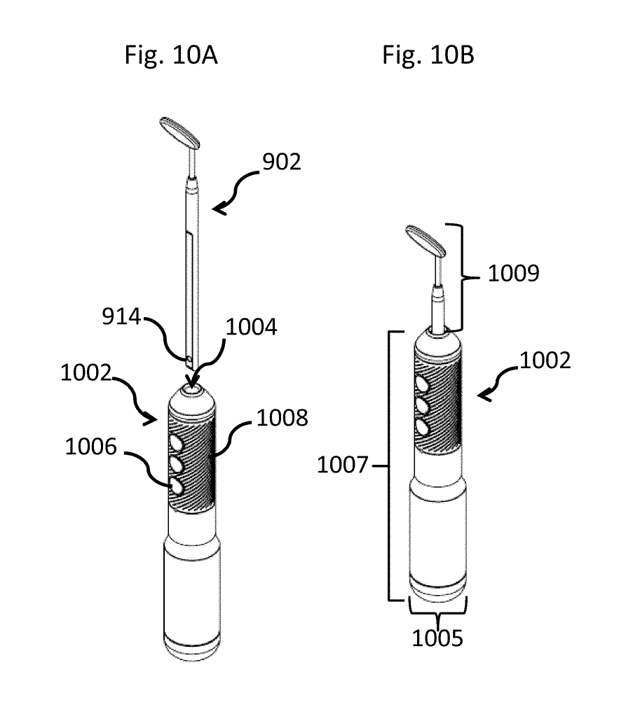

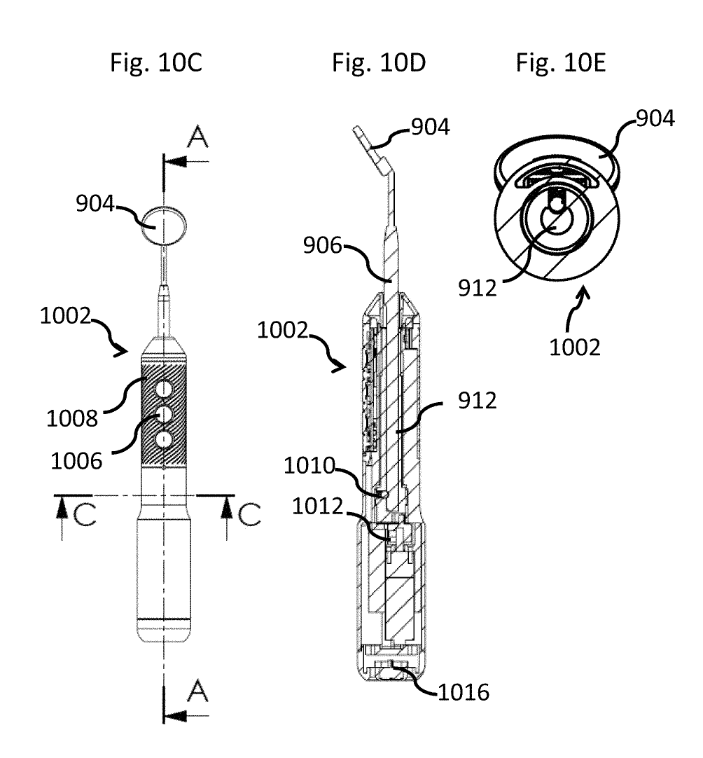

Reference is now made to FIGS. 10A-10E depicting a vibrating device coupled to a handle of a dental mirror, according to some exemplary embodiments of the device.

According to some exemplary embodiments, for example as shown in FIGS. 10A and 10B, a shaft, for example shaft 902 is inserted into a vibrating device, for example vibrating device 1002 through opening 1004. I n some embodiments, the vibrating device comprises an elongated body with a hand gripping portion 1008. In some embodiments, at least one button, for example an activating and/or operating button is positioned on the outer surface of the vibrating device body. Alternatively, the at least one button protrudes through an opening in the outer surface of the vibrating device body. Optionally, the at least one button is located at the gripping portion 1008 of the vibrating device body. In some embodiments, the at least one button is part of the interface 416 shown in FIG. 4.

According to some exemplary embodiments, at least part of the vibrating device, for example vibrating device e has a cylindrical shape. In some embodiments, the vibrating device diameter, for example diameter 1005 shown in FIG. 10B is in a range of 15-50 mm, for example 20 mm, 24 mm, 25 mm, 28 mm, 30 mm or any intermediate, larger or smaller diameter. In some embodiments, the axial length of the vibrating device, for example axial length 1007 shown in FIG. 10B is in a range of 10-30 cm, for example 12 cm, 15 cm, 20 cm or any intermediate, smaller or larger length.

According to some exemplary embodiments, the dental mirror extends to a distance, for example distance 1009 shown in FIG. 10B, of up to 30 cm from the vibrating device, for example the dental mirror extends to a distance of 10 cm, 15 cm, 20 cm or any intermediate, smaller or larger distance from the vibrating device.

According to some exemplary embodiments, for example as shown in FIG. 10D, a recess in the proximal section 912 of the handle fits a mechanical joint or a ball plunger, for example joint 1010 inside the vibrating device 1002. In some embodiments, joint 1010 is part of an interference fit mechanism of the vibrating device 1002 that prevents, for example the release of the handle from the vibrating device 1002 during activation and/or prevents relative movement between the handle and the vibrating device when the vibrating device is not activated.

According to some exemplary embodiments, the vibrating device 1002 comprises a gear mechanism 1012, optionally comprising a vibration mechanism mechanically coupled to the handle. Additionally, the vibrating device 1002 comprises an electrical charging socket 1016, shaped and sized to allow connection of a charging connector and/or a charging dock to the vibrating device.

According to some exemplary embodiments, for example as shown in FIG. 10E depicting a cross section along C-C line in the vibrating device 1002, at least a section of the handle, for example the proximal section has an asymmetrical cross section 912 as previously shown in FIG. 9D.

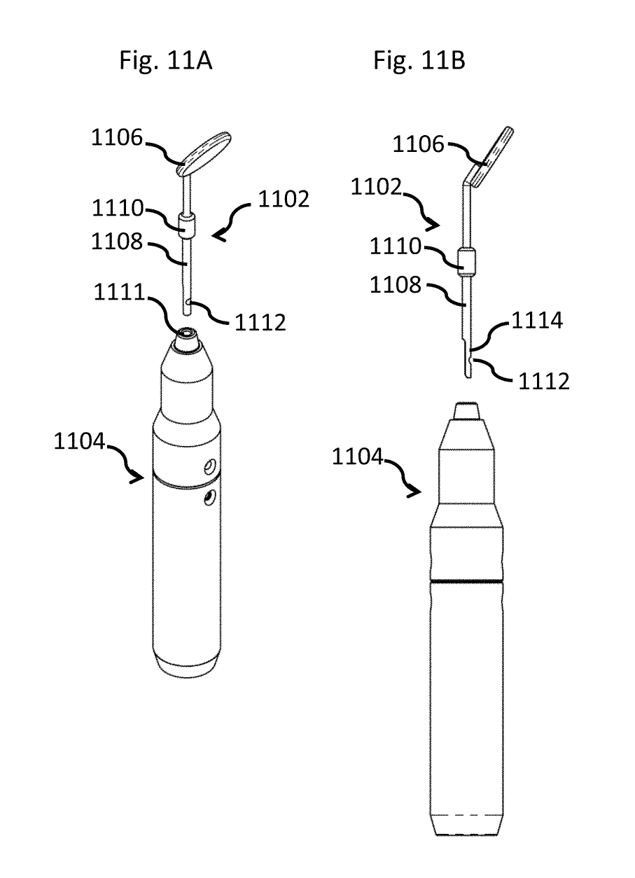

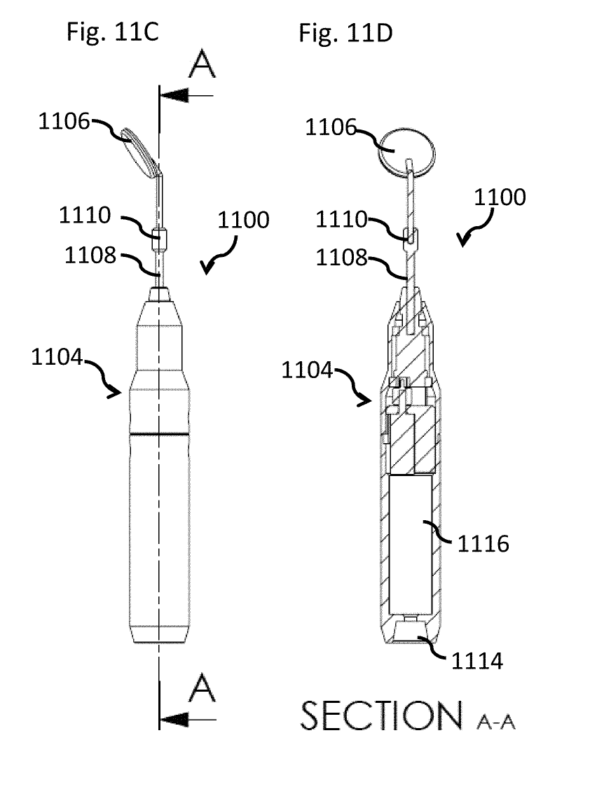

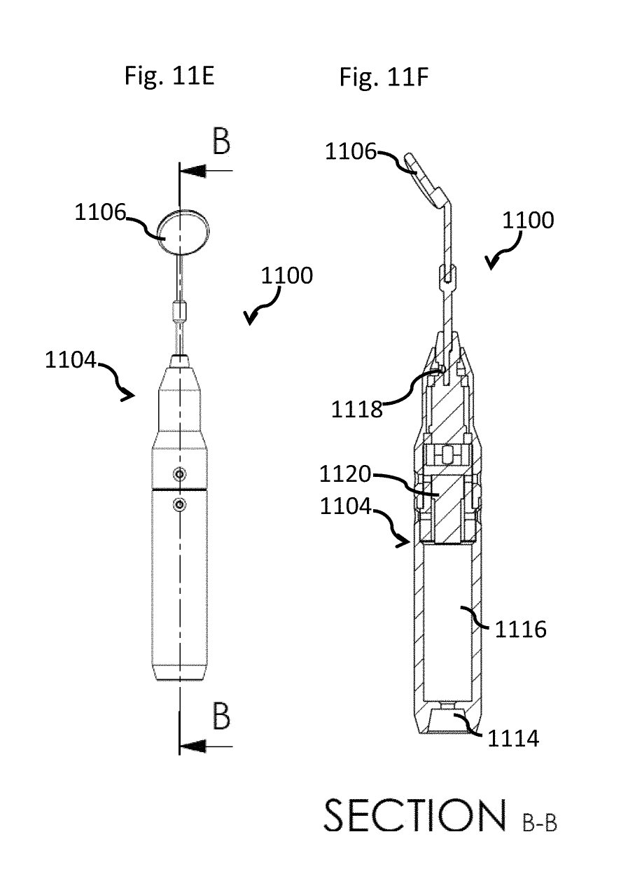

Reference is now made to FIGS. 11A-11F depicting a connection between a dental mirror and a vibrating device via an adaptor, according to some exemplary embodiments of the invention.

According to some exemplary embodiments, a dental mirror 1106 is connected to an adaptor 1108 through an adaptor connector 1110 located at the distal end of the adaptor 1108, for example the end of the adaptor 1108 near the dental mirror. Alternatively, the dental mirror 1106 and the adaptor are manufactures as a single non-separable unit. In some embodiments, the adaptor is shaped and sized to be inserted, at least partly into the vibrating device, for example vibrating device 1104 through an opening 1111. In some embodiments, the adaptor connector 1110 determines or is used to adjust the relative angle between the dental mirror 1106 and the adaptor 1108 or between the dental mirror 1106 and the vibrating device.

According to some exemplary embodiments, the adaptor 1108 comprises a recess 1112 located at a proximal section 1114 of the adaptor 1108, for example a section of the adaptor located at a distance from the dental mirror 1106 and/or from the adaptor connector 1110. In some embodiments, the recess 1112 fits an interference fit, optionally a mechanical joint inside the vibrating device 1104. In some embodiments, for example as shown in FIG. 11D, the proximal section 1114 is inserted up to 10 cm into the vibrating device, for example up to 9 cm, 7 cm, 5 cm or any intermediate, smaller or larger distance into the vibrating device.

According to some exemplary embodiments, for example as shown in FIGS. 11D and 11F, the vibrating device 1104 comprises an interference mechanism, for example a snap-fit mechanism, or a press fit mechanism. In some embodiments, the press-fit mechanism comprises a press-fit spring plunger or a press-fit ball plunger, for example ball plunger 1118. In some embodiments, the ball plunger 1118, locks the adaptor inside the vibrating device 1104. Optionally, the ball plunger 1118 fits at least partly into recess 1112 or to a different interlocking geometry, for example to mechanically interfere with the removal of the adaptor 1108 from the vibrating device 1104. In some embodiments, the interference mechanism, optionally comprising ball plunger 1118 mechanically interferes with recess 1112, for example to resist rotational movements generated by the vibrating device.

According to some exemplary embodiments, for example as shown in FIGS. 11D and 11F, the vibrating device 1104 comprises an electrical charging socket 1114, optionally at the base of the vibrating device. In some embodiments, the vibrating device comprises a power source, for example battery 1116 comprises an electrical power source, for example battery 1116 and an electric motor 1120 electrically connected to the battery 1116. Additionally, the electrical charging socket 1114 is electrically connected to the battery 1116.

### Exemplary Connection between Vibrating Device and a Charging Dock

According to some exemplary embodiments, the vibrating device comprises a replaceable power source, for example a replaceable battery. Alternatively or additionally, the power source is a rechargeable power source. In some embodiments, the vibrating device is connected to an electrical charger plug, for example to charge the power source of the vibrating device. Alternatively, the vibrating device is placed in a charging dock, optionally in an upright position. In some embodiments, a charging plug or a charging pin in the charging dock fits into a socket in the vibrating device, optionally into a socket positioned in the base of the vibrating device. Alternatively, a charging plug or a charging pin in the vibrating device fits into a socket in the charging dock.

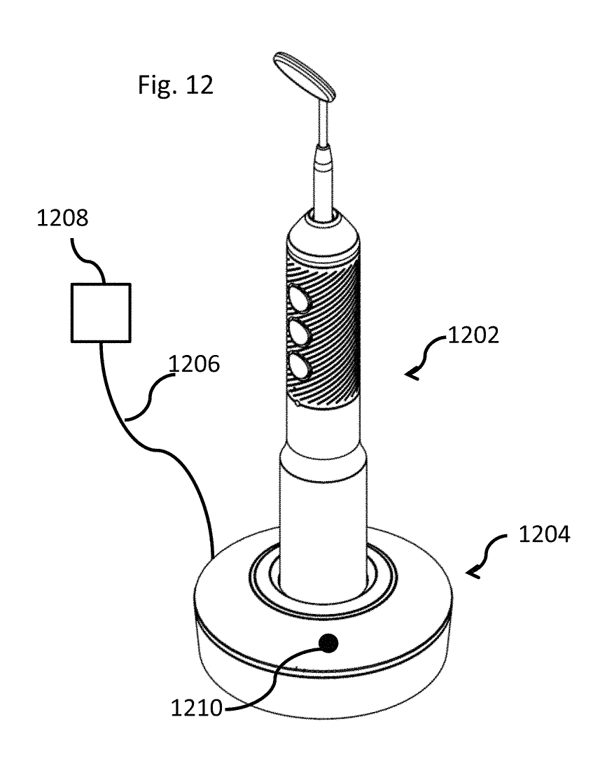

Reference is now made to FIG. 12, depicting a vibrating device positioned at least partly inside a charging dock, according to some exemplary embodiments of the invention.

According to some exemplary embodiments, the vibrating device 1202 is positioned at least partly inside charging dock 1202. In some embodiments, for example when the vibrating device is not in use and/or between treatment, the vibrating device 1202 is placed at least partly inside a charging dock 1204. In some embodiments, the charging dock is connected via electrical wiring 1206 to an electrical network 1208.

According to some exemplary embodiments, the charging dock 1204 comprises a lumen, optionally a central lumen with a size that fits around the bottom part of the vibrating device 1202. In some embodiments, the charging dock 1204 comprises at least one light indicator 1206 for delivering at least one light indication regarding the charging process of the vibrating device 1202. Alternatively or additionally, the at least one light indicator 1206 delivers at least one light indication regarding the electrical connection between the charging dock 1204 and the vibrating device 1202.

### Exemplary Vibrating Dental Device

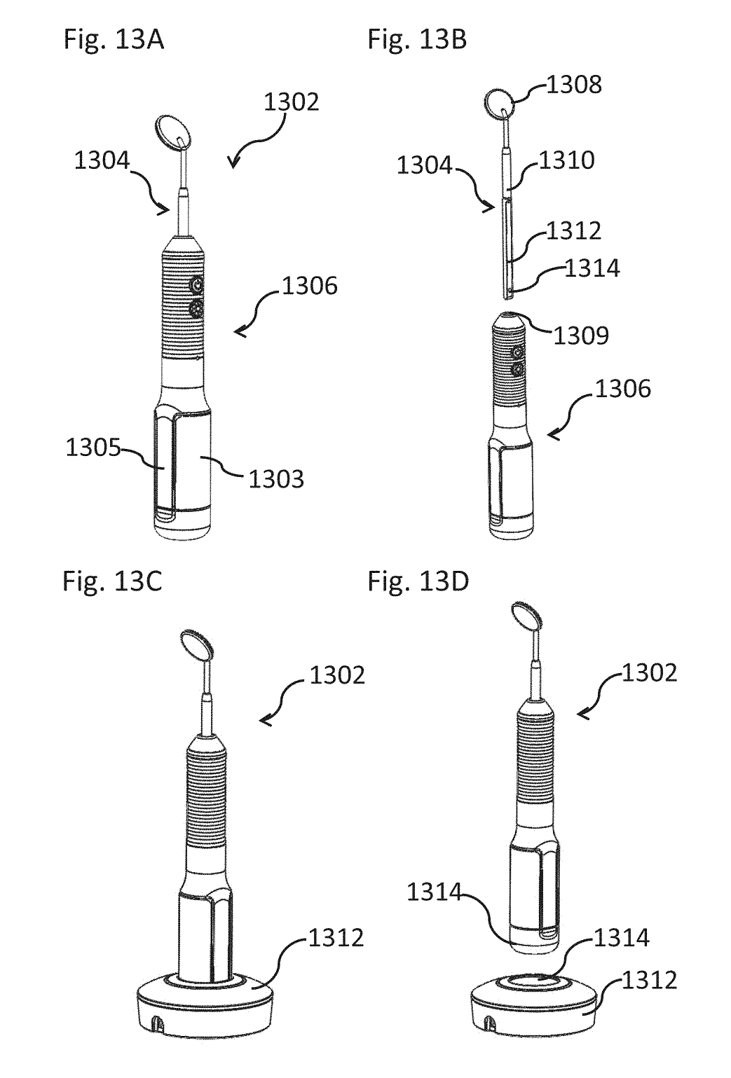

Reference is now made to FIGS. 13A-13D depicting a vibrating dental device, according to some exemplary embodiments of the invention.

According to some exemplary embodiments, a vibrating dental device, for example device 1302 comprises a dental mirror, for example dental mirror 1304 and a vibrating handle 1306. In some embodiments, the vibrating handle 1306 comprises a body 1303, for example a round shaped body. In some embodiments, the body 1303 comprises a curved outer surface. In some embodiments, at least one portion of the outer surface of the body 1303 is planar, for example planar portion 1305. Alternatively, at least one portion of the outer surface of the body 1303 comprises an indentation.

According to some exemplary embodiments, for example as shown in FIG. 13B, the dental mirror 1304 is pushed through an opening 1309 into the vibrating handle 1306. In some embodiments, the dental mirror 1304 comprises a mirror 1308 and a shaft, for example shaft 1310 having a distal section and a proximal section. The shaft 1310 is optionally an elongated shaft. In some embodiments, the distal section of the shaft 1310 is shaped and sized to fit through the opening 1309 and into an internal lumen within the vibrating handle.

According to some exemplary embodiments, a portion of the distal section, for example portion 1312 has an asymmetrical cross-section, for example a D-shaped cross section, for example to align the dental mirror 1302 relative to the vibrating handle. Optionally, the shaft 1310 of the dental mirror 1304 comprises a groove or a recess, for example recess 1314, or recess 914 shown in FIG. 9C. In some embodiments, the recess 1314 is positioned in the distal section on the shaft, and is sized and shaped to fit a press-fit locking mechanism, for example a ball plunger within the internal lumen of the vibrating handle 1306.

According to some exemplary embodiments, for example as shown in FIGS. 13C and 13D, the vibrating dental device 1302 is a cordless device. In some embodiments, the vibrating dental device 1302 comprises one or more replaceable batteries. In some embodiments, the vibrating dental device 1302 comprises one or more rechargeable batteries. In some embodiments, the vibrating dental device 1302 is configured to electrically charge the one or more rechargeable batteries by an electrical charger. Optionally, at least a portion of the vibrating dental device 1302 is shaped and sized to fit into a charging dock, for example charging dock 1312. Optionally, a base 1314 of the vibrating dental device 1302 is shaped and sized to fit through an opening 1314 of the charging dock 1312.

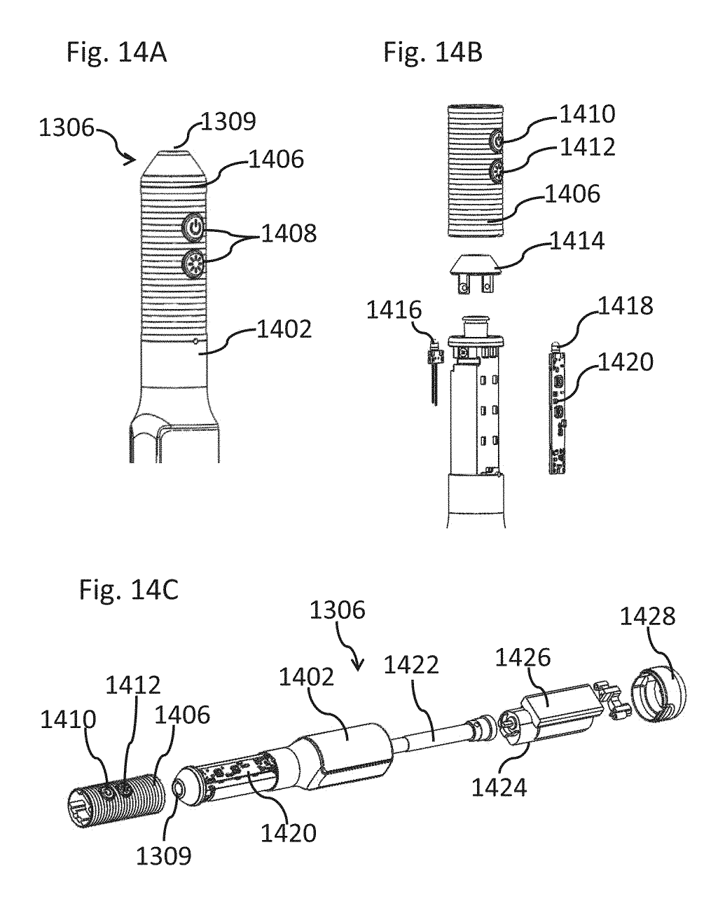

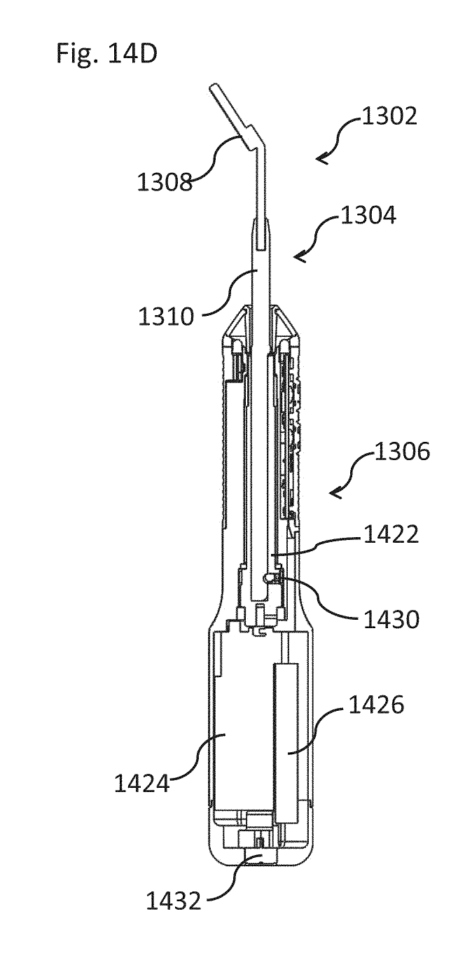

Reference is now made to FIGS. 14A-14C, depicting a vibrating handle, according to some exemplary embodiments of the invention.

According to some exemplary embodiments, a vibrating handle, for example vibrating handle 1306, comprises an elongated body 1402 having one or more gripping portions, for example gripping portion 1406. In some embodiments, the vibrating handle 1306, comprises an opening, for example opening 1309, which is shaped and sized to allow insertion of a dental mirror shaft into the vibrating handle, for example as described in FIGS. 13A-13D. In some embodiments, the vibrating handle body, for example body 1402 is made at least partly from plastic or metal.

According to some exemplary embodiments, the one or more gripping portions, for example gripping portion 1406 are positioned on the outer surface of the body 1402. Optionally, the gripping portion 1406 is a silicon gripping portion. In some embodiments, the gripping portion 1406 is a textured gripping portion, optionally containing a plurality of grooves.

According to some exemplary embodiments, the vibrating handle 1306 comprises a user interface, which optionally includes one or more activation buttons, for example activation buttons 1408. In some embodiments, the one or more activation buttons are positioned near the external surface of the body 1402. Optionally, the one or more activation buttons are positioned within the gripping portion 1406. In some embodiments, the one or more activation buttons allows activation of the dental vibrating device with and without vibration. In some embodiments, the activation buttons 1408 comprise a vibration and lighting activation button 1410, configured to activate vibration and lights of the vibrating dental device, simultaneously. Additionally, the activation buttons 1408 comprise lights activation button 1412, configured to activate the lights of the device independently from vibration activation. A potential advantage of having separate activation buttons for two activation modes of the device is that it allows a user to easily select a preferred activation mode of the device while treating a patient. For example, a user of the device, for example a dentist, selects a lights only activation mode while navigating to a desired contact site. Once reaching a desired contact site, the user selects to activate both lights and vibrations, for example to vibrate the contact site.

According to some exemplary embodiments, the vibrating handle comprises one or more light sources, for example one or more light-emitting diode (LED) sources. In some embodiments, the LED sources comprise LED sources 1416 and 1418. In some embodiments, the one or more light sources are configured to illuminate a contact site of the dental mirror with a tissue, for example a deformable tissue. Alternatively or additionally, the one or more light sources are configured to illuminate a target site, for example an injection target site. In some embodiments, light emitted from the one or more light sources is directed through a lens, for example lens 1414. Optionally, lens 1414 is a forward facing lens configured to direct the emitted light towards the dental mirror.

According to some exemplary embodiments, the vibrating handle comprises one or more printed circuit boards (PCBs), for example PCB 1420. In some embodiments, the one or more PCBs electrically connect the LED sources and/or the activation buttons of the vibrating handle to a power source of the vibrating dental device.

According to some exemplary embodiments, the vibrating handle comprises one or more motors, for example a pneumatic motor or an electrical motor. Alternatively, the vibrating handle is connected to an external motor, for example an electric motor or a pneumatic motor. Optionally, the vibrating handle is connected to a compresses air source.

According to some exemplary embodiments, the vibrating handle 1306 comprises at least one electric motor, for example motor 1424. In some embodiments, motor 1424 is mechanically coupled to a gear, for example an eccentric gear configured to transform rotational movement output of the motor 1424 into vibrations. In some embodiments, the gear is mechanically connected to a dental mirror connector, for example a dental mirror holder 1422, which is configured to hold and optionally to lock the dental mirror shaft within the vibrating handle.

Reference is now made to FIG. 14D, depicting a longitudinal cross-section of a vibrating dental device, according to some exemplary embodiments of the invention.

According to some exemplary embodiments, a vibrating dental device 1302 comprises a dental mirror 1304 removably coupled to a vibrating handle 1306. In some embodiments, the dental mirror 1304 is inserted through an opening in the vibrating handle into an internal lumen within the handle. In some embodiments, the dental mirror is inserted into a dental mirror holder 1422 within the lumen. In some embodiments, a press-fit locking mechanism within the vibrating handle is pressed into a recess within a shaft of the dental mirror, for example recess 1314 shown in FIG. 13B. In some embodiments, the press-fit locking mechanism comprises a ball plunger, for example ball plunger 1430 which is shape and sized to fit into the recess of the dental mirror shaft 1310.

It is expected that during the life of a patent maturing from this application many relevant dental vibrating devices will be developed; the scope of the term vibration device is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A vibrating dental device, comprising:
an elongated handle, wherein said handle further comprises a vibrator configured to generate vibrations with at least one desired vibration parameter;
a dental mirror connected to said vibrator, wherein an end of said dental mirror is sized and shaped to be placed in contact with a deformable soft tissue of a mouth at a contact site;
a driving circuitry within said elongated handle electrically connected to said vibrator, wherein said driving circuitry delivers electricity to said vibrator to vibrate said dental mirror with at least one vibration parameter selected to reduce sensitivity to pain at a different target site from said contact site, while said dental mirror is in contact with said deformable soft tissue at said contact site.

2. The device of claim 1, wherein said deformable soft tissue comprises the inner side of a cheek and/or the inner side of a lip.

3. The device of claim 2, wherein said dental mirror contacts said contact site at an angle to allow visualization of said target site.

4. The device of claim 1, wherein said selected at least one vibration parameter comprises vibration amplitude and/or vibration frequency.

5. The device of claim 4, wherein said vibration amplitude is at least 2 mm.

6. The device of claim 4, wherein said vibration frequency is at least 10 Hz.

7. The device of claim 1, further comprising a control circuitry, wherein said control circuitry controls said vibrator to generate said vibration according to said at least one selected vibration parameter.

8. The device of claim 7, wherein said control circuitry controls the delivery of said electricity to said vibrator by said driving circuitry.

9. The device of claim 1, further comprising a memory storage, wherein said memory storage stores at least one vibration protocol and/or at values of least one vibration parameter.

10. The device of claim 9, further comprising an interface for selecting said at least one vibration protocol and/or said values of said at least one vibration parameter.

11. The device of claim 1, wherein said handle further comprises a damper, wherein said damper reduces the vibrations delivered from said vibrator to said handle by at least 50% compared to the vibrations of said dental mirror.

12. The device of claim 1, further comprising a sensor coupled between said dental mirror and said vibrator, to sense a vibration parameter of said dental mirror.

13. The device of claim 12, wherein said vibration parameter of said dental mirror comprise vibration amplitude and/or vibration frequency.

14. The device of claim 1, wherein the distance between said contact site to said target site is at least 1 centimeter.

15. The device of claim 1, comprising a first interlocking geometry, and wherein said dental mirror comprises a second interlocking geometry, and wherein said first interlocking geometry and said second interlocking geometry are shaped and sized for mechanically interlocking with each other.

16. A method for reducing sensitivity to pain, comprising:
placing at least part of an attachment piece connected to a vibrating device in contact with a deformable tissue of a mouth at a contact site;
vibrating said deformable tissue using said attachment piece for reducing sensitivity to pain at a different target site from said contact site, wherein said target site is a remote target site located at a distance from said contact site.

17. The method of claim 16, wherein said attachment piece comprises a dental mirror.

18. The method of claim 17, wherein said deformable tissue of the mouth comprises an inner side of a cheek or an inner side of a lip, and said remote target site is located at a periodontal tissue.

19. The method of claim 18, wherein said remote target site is located at a distance of at least 1 cm from said contact site.

20. The device of claim 2, wherein said target site is located at a periodontal tissue.
